(19) United States
(12) Patent Application Publication
(10) Pub. No.: US 2025/0011862 A1
(43) Pub. Date: Jan. 9, 2025

(54) CAPTURING AND AMPLIFYING POLYNUCLEOTIDES USING PARTICLES

(71) Applicant: Illumina, Inc., San Diego, CA (US)

(72) Inventors: Sahngki Hong, San Diego, CA (US); Fei Shen, San Diego, CA (US); Lewis Kraft, Acworth, GA (US)

(73) Assignee: Illumina, Inc., San Diego, CA (US)

(21) Appl. No.: 18/753,228

(22) Filed: Jun. 25, 2024

Related U.S. Application Data
(60) Provisional application No. 63/510,875, filed on Jun. 28, 2023.

(51) Int. Cl. C12Q 1/6874; C12Q 1/6844

(52) U.S. Cl. CPC C12Q 1/6874; C12Q 1/6844

(57) ABSTRACT

In some examples, a method for capturing and amplifying a polynucleotide includes capturing the polynucleotide at a particle comprising a first region and a second region. The first region may include a first moiety that captures the polynucleotide. The second region may include a plurality of amplification primers and have a surface area which is substantially larger than the surface area of the first portion. The method includes using the plurality of amplification primers to amplify the captured polynucleotide.

### CROSS-REFERENCE TO RELATED APPLICATIONS

This application claims the benefit of U.S. Provisional Patent Application No. 63/510,875, filed Jun. 28, 2023 and entitled “Capturing and Amplifying Polynucleotides Using Particles,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2435-US.xml”, was created on Jun. 20, 2024 and is 16 kB in size.

### FIELD

This application generally relates to capturing and amplifying polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification (e.g., bridge amplification) are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to capturing and amplifying polynucleotides using particles. Particles for performing such capture and amplification, and methods of making such particles, also are disclosed.

Some examples herein provide a method for capturing and amplifying a polynucleotide. The method may include capturing the polynucleotide at a particle including a first region and a second region. The first region includes a first moiety that captures the polynucleotide. The second region includes a plurality of amplification primers and has a surface area which is substantially larger than the surface area of the first portion. The method includes using the plurality of amplification primers to amplify the captured polynucleotide.

In some examples, the captured polynucleotide inhibits capture of any other polynucleotide at the first region of the particle. In some examples, the surface area of the first portion inhibits capture of more than one polynucleotide.

In some examples, the first region has a dimension which is approximately equal to or smaller than a diameter of gyration of the polynucleotide. In some examples, the surface area of the first region is approximately 20 nm² or less. In some examples, the first region forms less than about 10% of an overall surface area of the particle.

In some examples, the first region is formed using a first material, and the second region is formed using a second material that is different than the first material. In some examples, at least one of the first and second regions includes a hydrogel.

In some examples, the first moiety includes a capture primer having a sequence that is orthogonal to sequences of the amplification primers. In some examples, the polynucleotide includes a single-stranded primer that is substantially complementary to, and hybridizes to, the capture primer. In some examples, the polynucleotide includes at least one adapter which is complementary to an amplification primer of the plurality of amplification primers.

In some examples, the first moiety covalently or non-covalently bonds to a second moiety coupled to the polynucleotide.

In some examples, the plurality of amplification primers includes a first type of amplification primers and a second type of amplification primers having a sequence that is orthogonal to a sequence of the first type of amplification primers. In some examples, the first type of amplification primers includes an excision moiety.

In some examples, the captured polynucleotide is double stranded. In some examples, the captured polynucleotide is amplified using strand invasion.

In some examples, the captured polynucleotide is single stranded. In some examples, the captured polynucleotide is amplified using bridge amplification. In some examples, the particle further includes oligonucleotides which are hybridized to respective amplification primers when the polynucleotide is captured.

In some examples, the method further includes contacting the particle with a substrate. In some examples, the method further includes electrostatically attracting the particle to the substrate. In some examples, the particle is contacted with the substrate after capturing the polynucleotide with the particle. In some examples, the particle is contacted with the substrate after amplifying the captured polynucleotide.

Some examples herein provide a method of sequencing a polynucleotide. The method may include capturing and amplifying the polynucleotide using any one of the above methods to generate amplicons of the polynucleotide coupled to the particle. The method may include capturing the particle in a region of a flowcell. The method may include sequencing the amplicons in the region of the flowcell.

Some examples herein provide a particle for seeding and amplifying a polynucleotide. The particle may include a first region including a first moiety to capture the polynucleotide; and a second region including a second material having a surface area which is substantially larger than the surface area of the first portion, and including a plurality of amplification primers.

In some examples, the first region has a dimension which is approximately equal to or smaller than a diameter of gyration of the polynucleotide. In some examples, the surface area of the first region is approximately 20 nm² or less. In some examples, the first region forms less than about 10% of an overall surface area of the particle.

In some examples, the first region is formed using a first material, and the second region is formed using a second material that is different than the first material. In some examples, at least one of the first and second regions includes a hydrogel.

In some examples, the first moiety includes a capture primer having a sequence that is orthogonal to sequences of the amplification primers. In some examples, the first moiety is to covalently or non-covalently bond to a second moiety coupled to the polynucleotide.

In some examples, the plurality of amplification primers includes a first type of amplification primers and a second type of amplification primers having a sequence that is orthogonal to a sequence of the first type of amplification primers. In some examples, the first type of amplification primers includes an excision moiety.

In some examples, the particle is in contact with a substrate. In some examples, the particle is electrostatically attracted to the substrate.

In some examples, the particle further includes oligonucleotides which are hybridized to respective amplification primers when the polynucleotide is captured.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1H schematically illustrate example structures and operations for capturing and amplifying a polynucleotide using a particle.

FIGS. 2A-2B schematically illustrate additional example structures that may be used to capture a polynucleotide using a particle.

FIGS. 3A-3C schematically illustrate an example manner in which a particle may be used to inhibit capture of more than one polynucleotide.

FIGS. 4A-4I schematically illustrate an example flow of operations for preparing a particle that may be used to capture and amplify a polynucleotide.

FIGS. 5A-5K schematically illustrate another example flow of operations for preparing a particle that may be used to capture and amplify a polynucleotide.

FIGS. 6A-6M schematically illustrate another example flow of operations for preparing a particle that may be used to capture and amplify a polynucleotide.

FIGS. 7A-7G schematically illustrate another example flow of operations for preparing a particle that may be used to capture and amplify a polynucleotide.

FIGS. 8A-8C schematically illustrate additional respective example flows of operations for preparing a particle that may be used to capture and amplify a polynucleotide.

FIGS. 9A-9D schematically illustrate additional example structures and operations for capturing and amplifying a polynucleotide using a particle.

FIGS. 10A-10J schematically illustrate another example flow of operations for preparing a particle that may be used to capture and amplify a polynucleotide.

FIGS. 11A-11E schematically illustrate another example flow of operations for preparing a particle that may be used to capture and amplify a polynucleotide.

### DETAILED DESCRIPTION

Examples provided herein are related to capturing and amplifying polynucleotides using particles. Particles for performing such capture and amplification, and methods of making such particles, also are disclosed.

Some examples herein relate to using particles to capture and amplify a polynucleotide. More specifically, the present particles may include a first region for use in capturing a polynucleotide, and a second region for use in amplifying the captured polynucleotide. The first region may have sufficiently small dimensions as to inhibit capture of more than one polynucleotide at the particle. For example, the first region may have a lateral dimension of about 20 nm or less. The polynucleotide that the first region captures may occupy a sufficient portion of the first region that the first region substantially may not capture another polynucleotide. For example, the polynucleotide may occupy at least about 50%, at least about 75%, at least about 90%, or about 100% of the first region, and even may extend beyond the first region. As such, the first region may be sized such that a polynucleotide that it captures may exclude other nucleotides from also being captured by the first region. The second region may have larger dimensions than the first region, and may be used to generate a plurality of amplicons of the polynucleotide that the first region captures. Because the first region may capture substantially only a single polynucleotide, the second region may generate amplicons substantially only of that single polynucleotide. As such, the particle may be used to generate a substantially monoclonal cluster of the captured polynucleotide. The cluster then may be sequenced, e.g., using sequencing by synthesis. Because the cluster may include substantially only amplicons of a single polynucleotide, the sequencing readout from that cluster may be expected to be high quality.

First, some terms used herein will be briefly explained. Then, some example particles and example methods for capturing and amplifying a polynucleotide, and methods of making such particles, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), peptide nucleic acid (PNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide (such as PNA), branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity. In some examples, a polymerase is used in combination with another enzyme, such as a recombinase and/or a single-strand binding protein (e.g., gp32).

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.” In nonlimiting examples, a capture primer may be or include DNA, RNA, PNA, modified DNA, modified RNA, or modified PNA.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible.

In some examples, a substrate is or includes a particle. As used herein, a “particle” is a small localized object which exists as a discrete unit in a given medium. In detail, the term refers to microscopic particles with sizes ranging from atoms to molecules, such as nanoparticle or colloidal particle. A particle may refer to a substrate that is sufficiently small that it may be located within a fluid (such as a liquid), that is, substantially surrounded on all sides by the fluid. As such, a particle may be carried by (moved by) the fluid in which it is located, for example by flowing the fluid from one location to another location. As explained elsewhere herein, particles can have a variety of shapes. When a particle is at least partially spherical, it may be referred to as a “bead.” In some examples, a first substrate (such as a flat or patterned surface within a flow cell) may be used to support a second substrate (such as a particle).

In some examples, a substrate includes a patterned surface. A “patterned surface” refers to an arrangement of different regions in or on an exposed layer of a substrate. For example, one or more of the regions may include feature(s) that can be used to attract and/or be coupled to a particle. The feature(s) can be separated by interstitial regions where such features are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface, into which particles respectively may be disposed. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used to attract particles for sequencing that may be stable over sequencing runs with a large number of cycles. In some examples, covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. The azido groups of the hydrogel material may be converted to, or coupled to, groups with a positive charge. A solution including the present particles may then be contacted with the polished substrate such that individual target particles may become attracted to, and disposed within, individual wells via interactions with the positively charged groups attached to the hydrogel material; however, the particles substantially will not occupy the interstitial regions due to absence or inactivity of the hydrogel material. Before or after contacting the particles with the substrate, the particles may be used to capture and amplify a plurality of target polynucleotides (e.g., a fragmented human genome or portion thereof). Amplicons of the target polynucleotides may be confined to the wells because the particles are confined to the wells. The amplicons then may be sequenced. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate. A hydrogel may be located on a substrate, such as on a region of a particle or on a region of a flow cell.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides coupled to respective particles) that are at different regions of an array may be differentiated from each other according to the locations of the regions in the array. An individual region of an array may include one or more molecules of a particular type. For example, a substrate region may include a single particle including amplicons of a target polynucleotide having a particular sequence. The regions of an array respectively may include different features than one another on the same substrate. Exemplary features include without limitation, wells in a substrate, particles (such as beads) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or hydrogel. Exemplary arrays in which separate substrates are located on a surface include, without limitation, those having particles (such as beads) in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either 3′ end or 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide. The action by which a polynucleotide in solution becomes coupled to a substrate may be referred to as “capturing” the polynucleotide.

### Methods for Capturing and Amplifying Polynucleotides Using Particles

Some examples provided herein relate to generating clusters that are substantially monoclonal, by using particles that can capture substantially a single polynucleotide and then are used to amplify that polynucleotide to generate a cluster which is at least functionally monoclonal, and in some examples is substantially monoclonal.

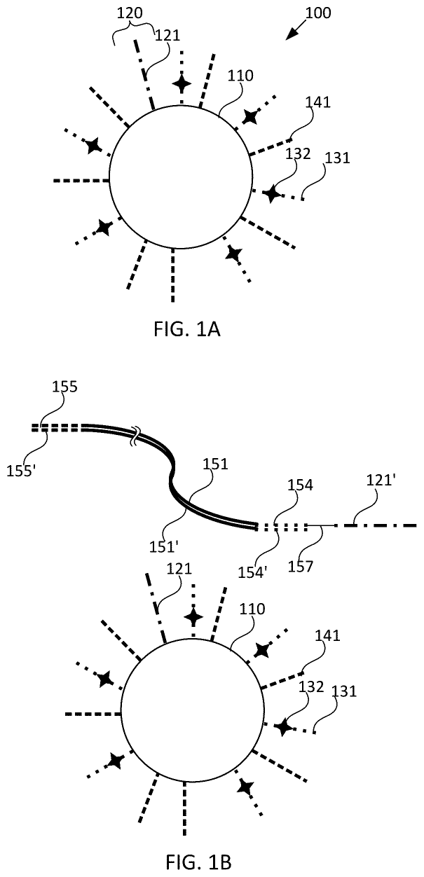

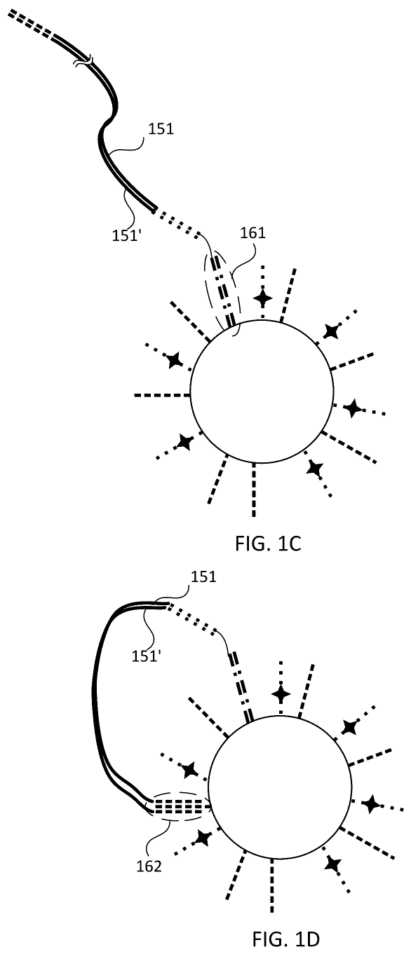

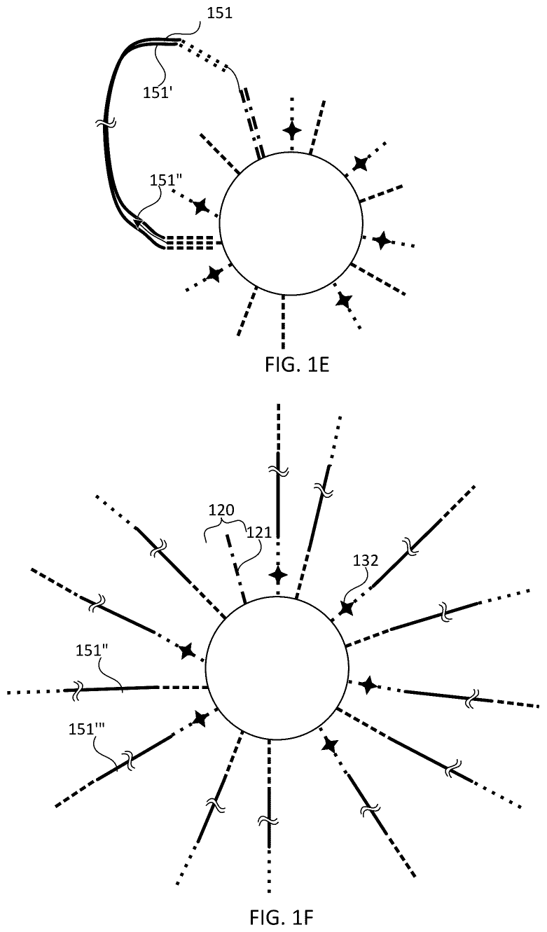

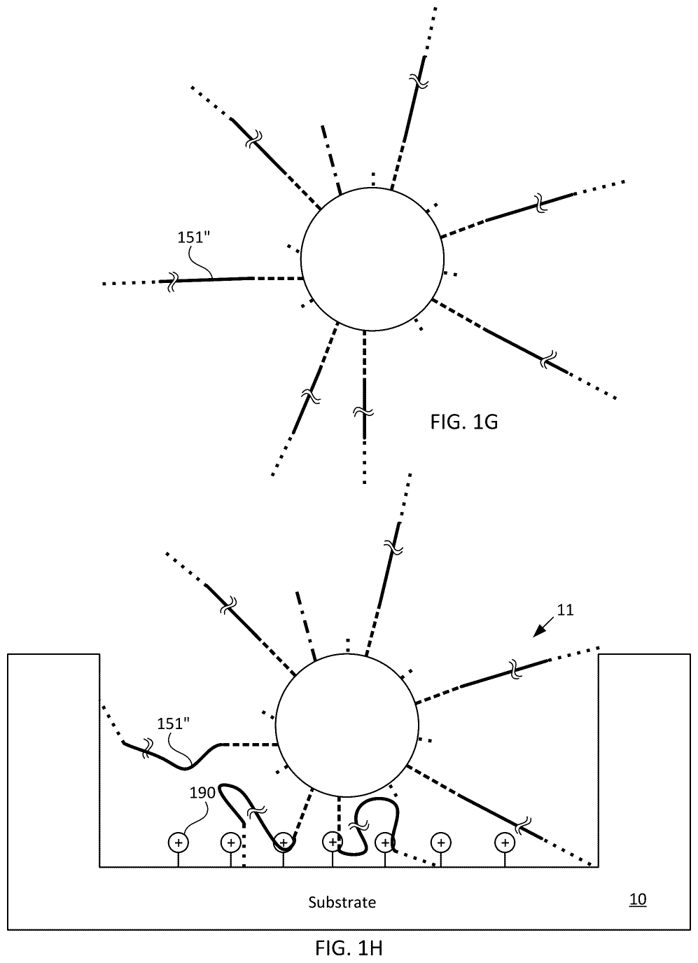

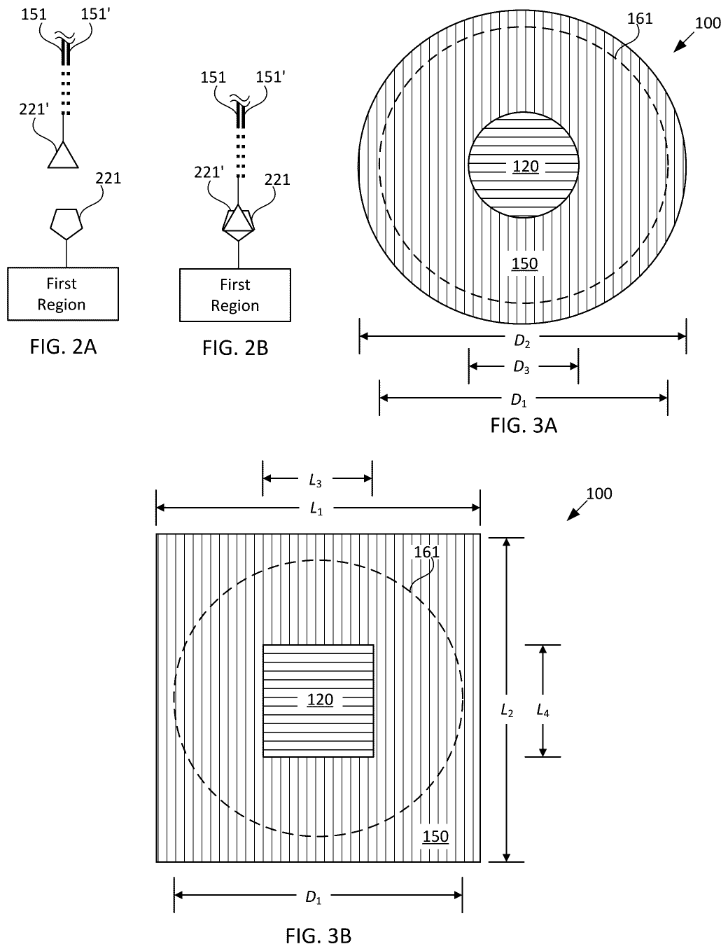

For example, FIGS. 1A-1H schematically illustrate example structures and operations for capturing and amplifying a polynucleotide using a particle. Referring first to FIG. 1A, particle 100 may include first and second amplification primers 131, 141 which have orthogonal sequences to one another. In the nonlimiting example illustrated in FIG. 1A, particle 100 may include one or more seeding primer(s) 121 which may have an orthogonal sequence to that of amplification primers 131, 141, and which may be used to capture a polynucleotide having an adapter which is complementary to seeding primer 121 in a manner such as will be described with reference to FIGS. 1B-1C. In other examples such as will be described with reference to FIGS. 2A-2B, instead of including one or more seeding primer(s), particle 100 may include a moiety that may be used to react with a moiety which is coupled to the polynucleotide.

Particle 100, illustrated in FIG. 1A, may include core 110 to which the seeding primer(s) 121 (if used) and amplification primers 131, 141 selectively may be coupled, e.g., using operations such as described elsewhere herein. In one nonlimiting example, capture primers 131 may include P5, capture primers 141 may include P7, and seeding primer(s) 121 may include PX or PY. The seeding primer(s) 121 may be located in a first region 120 of particle 100, and the first and second amplification primers 131, 141 may be located in a second region of the particle in which a mixture of first and second amplification primers is located. The first region 120 may form a relatively small fraction of the overall surface area of particle 100, e.g., about 1-10%, or less than about 10%, or less than about 5%, or less than about 2%, or less than about 1%, of the surface area of particle 100. In one nonlimiting example, particle 100 has a diameter of about 200 nm or less, and first region has a diameter of about 20 nm or less. Although only a single seeding primer 121 is shown in the illustrated example, first region 120 may include a plurality of seeding primers 121. However because the first region 120 may form a relatively small fraction of the overall surface area of particle 100, the seeding primers 121 may form a relatively small fraction of the overall number of primers that are coupled to particle 100, e.g., about 1-10%, or less than about 10%, or less than about 5%, or less than about 2%, or less than about 1%, of the total number of primers that are coupled to particle 100.

At the particular time illustrated in FIG. 1A, particle 100 may be located within a fluid (fluid not specifically illustrated), such an aqueous buffer solution. Referring now to FIG. 1B, particle 100 may be contacted, in the fluid, with double-stranded target polynucleotides, e.g., double-stranded polynucleotide fragments that are generated using commercially available fragmentation or tagmentation techniques, using DNA or RNA that it is desired to sequence. Although only one particle 100 is illustrated in FIG. 1B, it will be appreciated that the fluid may include thousands, or even millions, of particles that have substantially the same configuration as one another. Additionally, although only one double-stranded polynucleotide 151, 151′ is illustrated in FIG. 1B, it will be appreciated that the fluid may include thousands, or even millions, of polynucleotides which in some cases may have different lengths than one another. In the nonlimiting example illustrated in FIG. 1B, each of the polynucleotides 151, 151′ may include first and second adapters, e.g., first adapter 154, 154′ of which adapter 154 is substantially complementary to amplification primer 131, e.g., is or includes cP5 (also referred to as P5′); and second adapter 155, 155′ of which adapter 154 is substantially complementary to amplification primer 141, e.g., is or includes cP7 (also referred to as P7′). The double-stranded polynucleotides may include an additional single-stranded seeding adapter which may be used to constrain the location at which the polynucleotide may be immobilized to the particle during the seeding process. For example, as shown in FIG. 1B, polynucleotide 151, 151′ optionally may include single-stranded seeding adapter 121′ which is substantially complementary to seeding primer(s) 121, and which optionally is coupled to adapter 154 of polynucleotide 151 via linker 157. In some examples, optional seeding adapter 121 may include cPX (also referred to as PX′) or cPY (also referred to as PY′). Note that because it is single-stranded, optional seeding adapter 121′ is available to hybridize to optional seeding primer 121 in a manner such as now will be described.

In some examples, linker 157 may be or include one or more of: a carbon-containing chain with a formula (CH₂)ₙ wherein “n” is from 1 to about 1500, for example less than about 1000, preferably less than 100, e.g. from 2-50, particularly 5-25; polyethylene glycol (PEG); or iSp9 (Spacer 9) which is a triethylene glycol spacer that can be incorporated at 5′-end or 3′-end of an oligo or internally. Linkers formed primarily from chains of carbon atoms or from PEG may be modified so as to contain functional groups which interrupt the chains. Examples of such groups include ketones, esters, amines, amides, ethers, thioethers, sulfoxides, sulfones. Separately or in combination with the presence of such functional groups, groups such as alkene, alkyne, aromatic or heteroaromatic moieties, or cyclic aliphatic moieties (e.g., cyclohexyl) may be included. Cyclohexyl or phenyl rings may, for example, be connected to a PEG or (CH₂)ₙ chain through their 1- and 4-positions. Other linkers are envisaged which are based on nucleic acids or monosaccharide units (e.g., dextrose). It is also within the scope of this disclosure to utilise peptides as linkers. A variety of other linkers may be employed. The linker preferably is stable under conditions under which the polynucleotides are intended to be used subsequently, e.g., conditions used in DNA amplification. The linker should also be such that it is not by-passed by DNA polymerases, terminating DNA polymerization before copying adapter 121′ (if it is nucleotide based such as a PX′ sequence). This allows for the adapter 121′ to remain single-stranded and available for hybridization in a manner such as will now be described.

Referring now to FIG. 1C, seeding adapter 121′ of double-stranded polynucleotide 151, 151′ may hybridize to seeding primer 121 to form duplex 161. Because adapters 154, 154′ and 155, 155′ are double-stranded, they are not available to hybridize to adapters 131 and 141 respectively until after certain processing steps are performed, as will be explained below. Accordingly, substantially all seeding of the polynucleotide may be expected to be via hybridization between seeding adapter 121′ and seeding primer 121 in the first region of the particle 100. Additionally, because the seeding primer(s) 121 are concentrated in the first region 120, substantially all seeding may be limited to occurring within that region. These limitations may be used to inhibit particle 100 from capturing another polynucleotide.

For example, after the initial hybridization described with reference to FIG. 1C, polynucleotide 151, 151′ may be amplified relatively quickly. More specifically, as illustrated in FIG. 1D, the double-stranded polynucleotide 151, 151′ may bend such that adapter 155, 155′ hybridizes with one of primers 141 to form triplex 162 using a process that may be referred to as “strand invasion” and may be promoted using a recombinase (not specifically illustrated in FIG. 1D). An amplified cluster then may be formed using polynucleotide 151, 151′. For example, FIG. 1E illustrates the composition of FIG. 1D during recombinase-promoted extension of the primer 141 to which double-stranded polynucleotide 151, 151′ hybridizes to form amplicon 151″ which is covalently coupled to the second region of the particle 100. Amplicon 151″ repeatedly may be further amplified using strand invasion. For example, it may be seen that the composition of FIG. 1F includes amplicon 151″ and a plurality of additional amplicons 151′″ of amplicon 151″ that are formed using a mixture of capture primers 131 and 141, which are located in the second region of the particle, for the amplification, while amplicons substantially may not be present in the first region 120 of the particle.

Amplification operations may be performed any suitable number of times so as to substantially fill the second region of the particle with an at least functionally monoclonal cluster, and in some examples a substantially monoclonal cluster, e.g., with amplicons of target polynucleotide 151, 151′. For example, amplicons within the second region of the particle may include at least about 60% amplicons of one selected target polynucleotide, or at least about 70% amplicons of one selected target polynucleotide, or at least about 80% amplicons of one selected target polynucleotide, or at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide. If amplification operations are repeated until the second region of the particle is substantially full, both adapters of the resulting amplicons may not necessarily be hybridized to corresponding capture primers or orthogonal capture primers, and as such the amplicons may extend linearly away from the particle 100 as illustrated in FIG. 1F. For further details regarding seeding and amplification operations using strand invasion and seeding adapters and seeding primers that are orthogonal to capture adapters and capture primers, see International Patent Application No. PCT/US2022/053002, filed on Dec. 15, 2022 and entitled “Orthogonal Hybridization,” the entire contents of which are incorporated by reference herein.

In some examples, certain capture primers and orthogonal capture primers may include non-nucleotide moieties. Such non-nucleotide moieties may include, but are not limited to, excision moieties via which a portion of the capture primers selectively may be removed. For example, capture primers 131 optionally may include excision moieties 132 and/or capture primers 141 optionally may include excision moieties (excision moieties of capture primers 141 not specifically shown in FIGS. 1A-1F). The excision moieties 132 of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following a desired number of amplification operations such as described with reference to FIGS. 1A-IF, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Excision moieties 132 may be used to remove amplicons that are oriented in a selected direction, so that the remaining amplicons are oriented substantially in the same direction as one another in a manner such as illustrated in FIG. 1G.

In some examples, following the seeding and amplification operations, particle 100 with amplicons 151″ coupled thereto may be disposed within a flowcell and the amplicons sequenced, e.g., using sequencing-by-synthesis. Illustratively, as shown in FIG. 1H, a substrate 10 (e.g., a surface of a flowcell) may include a plurality of positively charged moieties 190. In the nonlimiting example illustrated in FIG. 1H, the substrate 10 includes a recess 11 in which the positively charged moieties 190 are disposed. Optionally, the positively charged moieties may be coupled to a hydrogel (not specifically illustrated) which is located on the substrate (e.g., within the flowcell). The amplicons 151″ may have a negative charge that is electrostatically attracted to the positively charged moieties 190. This electrostatic attraction may draw particle 100 into contact with the substrate. Additionally, this electrostatic attraction and/or other force(s) may retain particle 100 in contact with the substrate 10. The amplicons 151″ then may be sequenced, e.g., using sequencing-by-synthesis operations that use reagents which may be flowed across the substrate 10 (sequencing operations not specifically illustrated).

In examples such as described with reference to FIGS. 1A-1H, the initial polynucleotide 151, 151′ is coupled to particle 100 via hybridization between primer 121 (which may be considered a first moiety) and adapter 121′ (which may be considered a second moiety that couples to the first moiety via hybridization). In these examples, primer 121 may be orthogonal to primers 131 and 141, as intended to be represented by the different line types used to illustrate primers 121, 131, and 141. However, hybridization between an adapter and a primer is only one example of a manner in which the initial polynucleotide may be coupled to the particle.

For example, FIGS. 2A-2B schematically illustrate example structures that may be used to capture a polynucleotide using a particle. As shown in FIG. 2A, the first region of the particle may include one or more first moiet(ies) 221, and the polynucleotide 151, 151′ may include a second moiety 221′ that is to couple to one of the first moiet(ies) so as to couple the polynucleotide to the first region in a manner such as illustrated in FIG. 2B. In some examples, moieties 221 and 221′ couple to one another via a noncovalent interaction (which also may be referred to as noncovalent bonding). Illustratively, the noncovalent interaction may be avidin-biotin interaction. For example, first moiety 221 may include a biotin moiety that allows for non-covalent bonding with a respective streptavidin binding site located on second moiety 221′. Or, for example, second moiety 221′ may include a biotin moiety that allows for non-covalent bonding with a respective streptavidin binding site located on first moiety 221. A non-exclusive list of complementary binding partners that may be used to non-covalently bond moiety 221 to moiety 221′ is presented in Table 1:

TABLE 1

| | Example moiety | Example moiety |
|---|---|---|
| Bonding pair | 221 or 221′ | 221′ or 221 |
| biotin-streptavidin | biotin, | Streptavidin, |
| | desthiobiotin, dual- | neutravidin, avidin, |
| | biotin, Strep-tag | Strep-Tactin |
| His-tag transition | transition metal | Histidine tag (His-tag) |
| metal | (e.g., Mn²⁺, Fe²⁺, | |
| | Co₂₊, Ni²⁺, or Cu²⁺) | |
| His-tag transition | His-tag | transition metal (e.g., |
| metal | | Mn²⁺, Fe²⁺, Co₂₊, Ni²⁺, |
| | | or Cu²⁺) |
| DIG/anti-DIG | digoxigenin (DIG) | anti-digoxigenin (anti- |
| | | DIG) antibody |
| c-myc/anti-cmyc | c-myc (also referred | anti-cmyc antibody |
| | to as MYC) | |
| c-myc/anti-cmyc | anti-cmyc antibody | c-myc |
| GST/glutathione | glutathione | glutathione s-transferase |
| | | (GST) |
| GST/glutathione | glutathione s- | glutathione |
| | transferase (GST) | |
| FLAG/anti-FLAG | FLAG tag | Anti-FLAG antibody |
| FLAG/anti-FLAG | Anti-FLAG | FLAG tag |
| | antibody | |

In other examples, moieties 221 and 221′ couple to one another using covalent bonding. In some such examples, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

Illustratively, moiety 221 may include one or more amino bonding sites, carboxy bonding sites, thiol bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxoamine bonding sites, SpyTag bonding sites, Snap-tag bonding sites, CLIP-tag bonding sites, or proteins with N-terminus recognized by sortase, or combinations thereof. In some such examples, moiety 221′ includes a functional moiety that allows for covalent bonding with moiety 221, illustratively a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. A non-exclusive list of complementary binding partners that may be used to covalently bond moiety 221 to moiety 221′ is presented in Table 2:

TABLE 2

| Bonding pair | Example moiety 221 or 221′ | Example moiety 221′ or 221 |
|---|---|---|
| amine-NHS | amine group, —NH₂ | 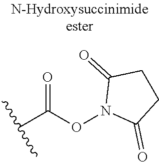 [SMILES: *C(=O)ON1C(=O)CCC1=O] |
| amine-imidoester | amine group, —NH₂ | 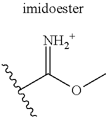 [SMILES: *C(=[NH2+])OC] |
| amine-pentofluorophenyl ester | amine group, —NH₂ | 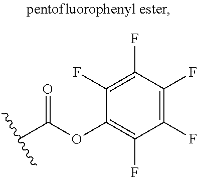 [SMILES: *C(=O)Oc1c(F)c(F)c(F)c(F)c1F] |
| amine-hydroxymethyl phosphine | amine group, —NH₂ | 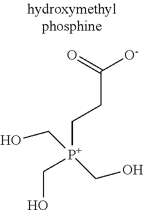 [SMILES: O=C([O-])CC[P+](CO)(CO)CO] |
| amine-carboxylic acid | amine group, —NH₂ | carboxylic acid group, —C(═O)OH (e.g., |
| | | following activation of the carboxylic acid |
| | | by a carbodiimide such as EDC (1-ethyl- |
| | | 3-(-3-dimethylaminopropyl)carbodiimide |
| | | hydrochloride) or DCC(N′,N′-dicyclohexyl |
| | | carbodiimide) to allow for formation of an |
| | | amide bond of the activated carboxylic acid |
| | | with an amine group) |
| thiol-maleimide | thiol, —SH | 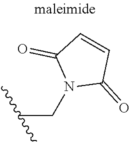 [SMILES: *CN1C(=O)C=CC1=O] |
| thiol-haloacetyl | thiol, —SH | 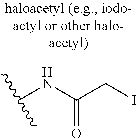 [SMILES: *NC(=O)CI] |
| thiol-pyridyl disulfide | thiol, —SH | 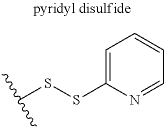 [SMILES: *SSc1ccccn1] |
| thiol-thiosulfonate | thiol, —SH | 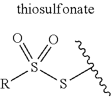 |
| thiol-vinyl sulfone | thiol, —SH | 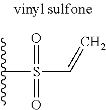 [SMILES: *S(=O)(=O)C=C] |
| aldehyde-hydrazide | aldehyde, —C(═O)H | 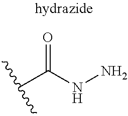 [SMILES: *C(=O)NN] |
| aldehyde-alkoxyamine | aldehyde, —C(═O)H | 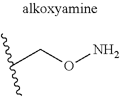 [SMILES: *CON] |
| hydroxy-isocyanate | hydroxyl, —OH | 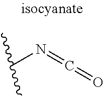 [SMILES: *N=C=O] |
| azide-alkyne | azide, —N₃ | 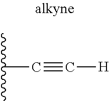 [SMILES: *C#C] |
| azide-phosphine | azide, —N₃ | 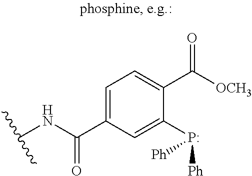 |
| azide-cyclooctyne or azide-cyclononyne | azide, —N₃ | 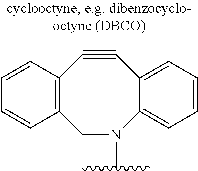 [SMILES: *N1Cc2ccccc2C#Cc2ccccc21] |
| | | 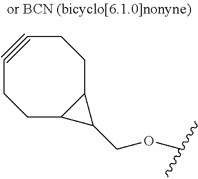 [SMILES: *OCC1C2CCC#CCCC21] |
| azide-norbornene | azine, —N₃ | 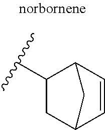 [SMILES: *C1CC2C=CC1C2] |
| transcyclooctene- tetrazine | 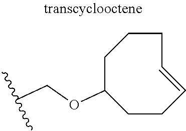 [SMILES: *COC1CC/C=C/CCC1] | 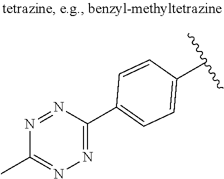 [SMILES: *c1ccc(-c2nnc(C)nn2)cc1] |
| norbornene-tetrazine | 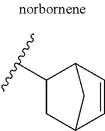 [SMILES: *C1CC2C=CC1C2] | 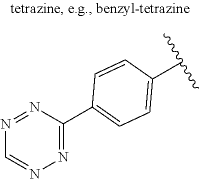 [SMILES: *c1ccc(-c2nncnn2)cc1] |
| oxime | aldehyde or ketone | alkoxyamine |
| | (e.g., amine cugroup or N- | |
| | terminus of polypeptide | |
| | converted to an aldehyde | |
| | or ketone by pyroxidal | |
| | phosphate) | |
| SpyTag-SpyCatcher | SpyTag: amino acid sequence | SpyCatcher amino acid sequence: |
| | AHIVMVDAYKPTK | MKGSSHHHHHHVDIPTTENLYFQGAM |
| | (SEQ ID NO: 9) | VDTLSGLSSEQGQSGDMTIEEDSATHIK |
| | | FSKRDEDGKELAGATMELRDSSGKTIS |
| | | TWISDGQVKDFYLYPGKYTFVETAAPD |
| | | GYEVATAITFTVNEQGQVTVNGKATK |
| | | (SEQ ID NO: 10) |
| SNAP-tag-O⁶- Benzylguanine | SNAP-tag (O—6-methyl- guanine-DNA methyl- transferase) | 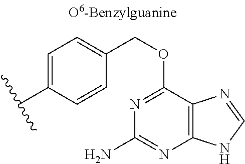 [SMILES: *c1ccc(COc2nc(N)nc3[nH]cnc23)cc1] |
| CLIP-tag-O²- benzylcytosine | CLIP-tag (modified O—6-methylguanine- DNA methyltransferase) | 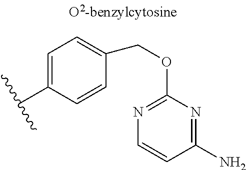 [SMILES: *c1ccc(COc2nccc(N)n2)cc1] |
| Sortase-coupling | -Leu-Pro-X-Thr-Gly | -Gly₍₃₋₅₎ (SEQ ID NO: 12) (SEQ ID NO: 13) |
| | (SEQ ID NO: 11) | |

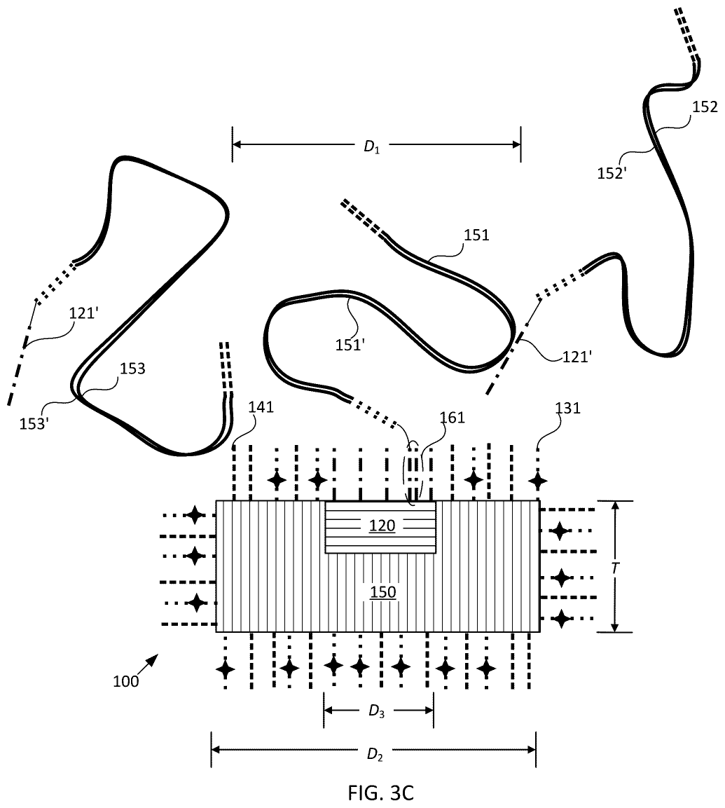

In some examples, the capture of a first polynucleotide 151, 151′ at the first region 120 of particle 100 may sterically inhibit other such polynucleotides from being able to become stably coupled to that region. For example, first polynucleotide 151, 151′ may occupy a sufficiently large portion of the first region 120 of the particle 100, such that another polynucleotide may not be able to access the first region in such a manner as to become coupled to the first region. Illustratively, FIGS. 3A-3C schematically illustrate an example manner in which a particle may be used to inhibit capture of more than one polynucleotide. More specifically, FIG. 3A illustrates a plan view of an example particle 100 that is shaped generally spherically or cylindrically, such that the first region 120 of the particle is substantially circular in the lateral dimension and has a diameter of D₃, and the second region 150 of the particle also is substantially circular in the lateral dimension and has a diameter of D₂. As such, in the lateral dimension shown in FIG. 3A, the surface area A₃ of the first region 120 may be approximately expressed as

$${A}_{3} = π {( \frac{{D}_{3}}{2} )}^{2} ,$$

and the surface area A₂ of the second region 150 may be approximately expressed as

$${A}_{2} = π {( \frac{{D}_{2}}{2} )}^{2} .$$

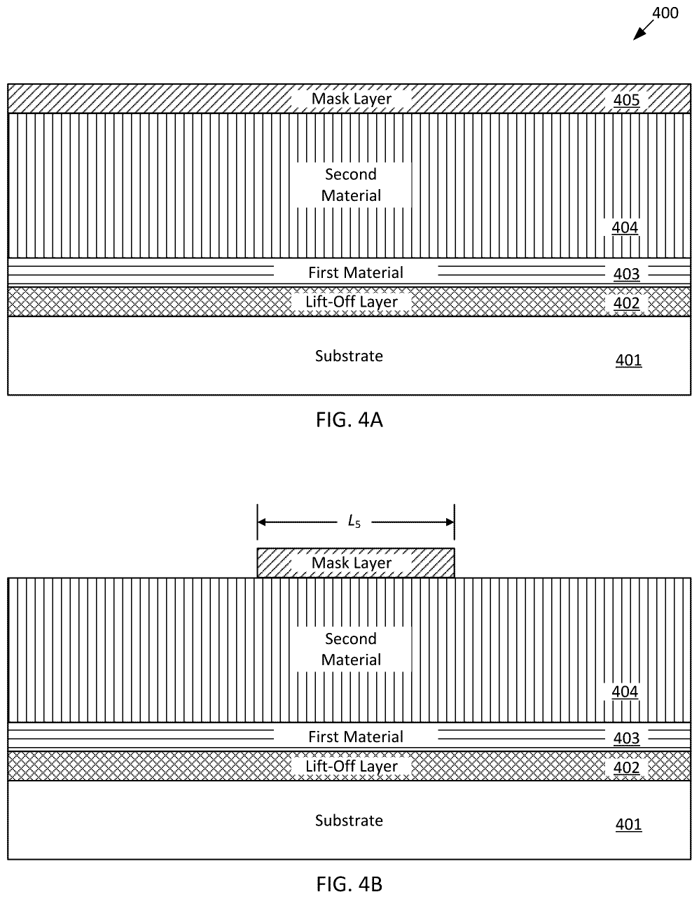

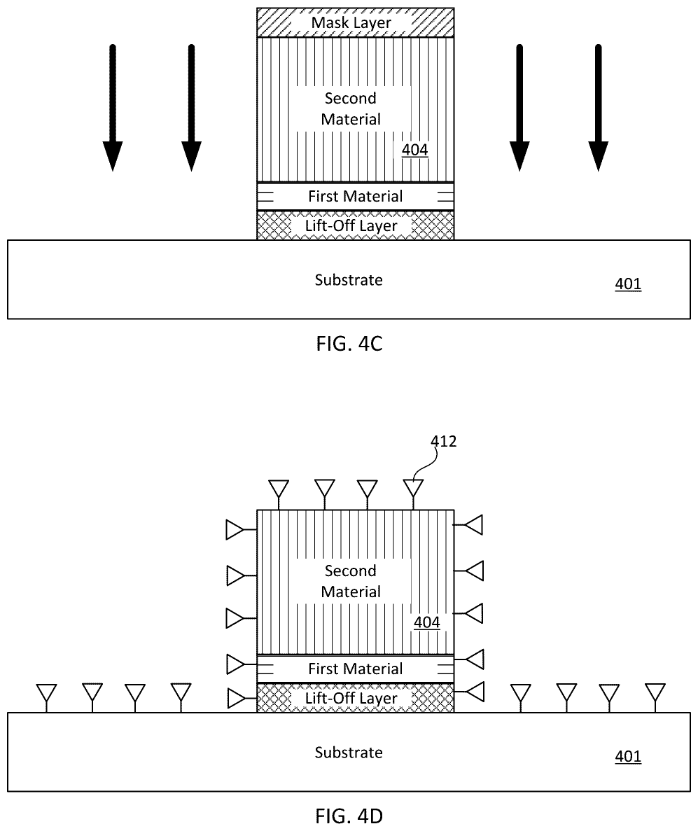

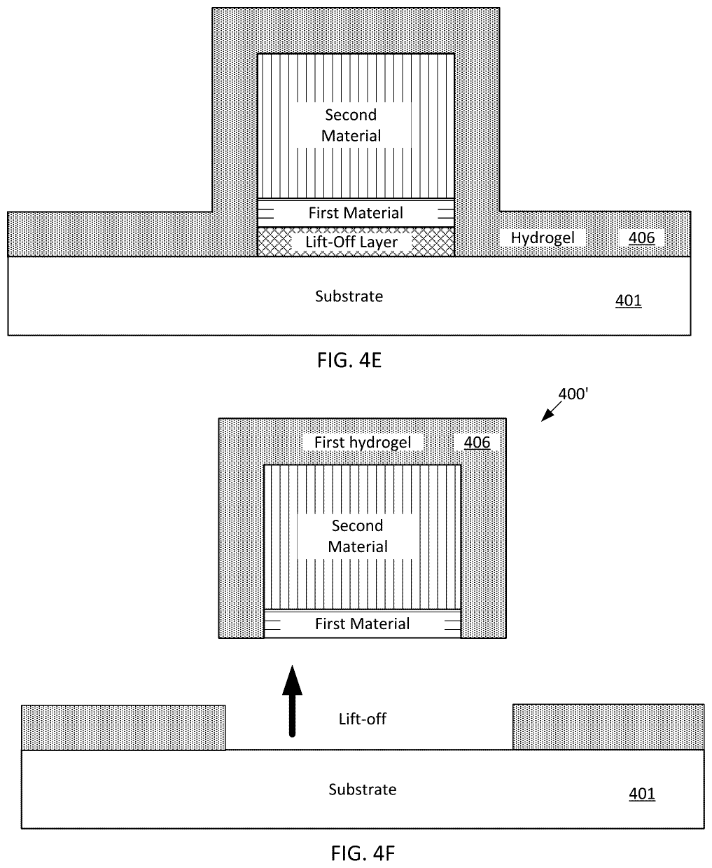

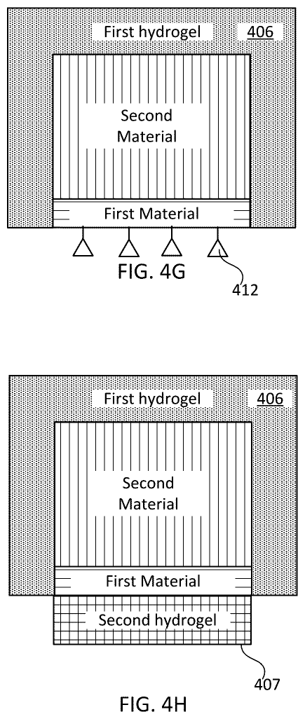

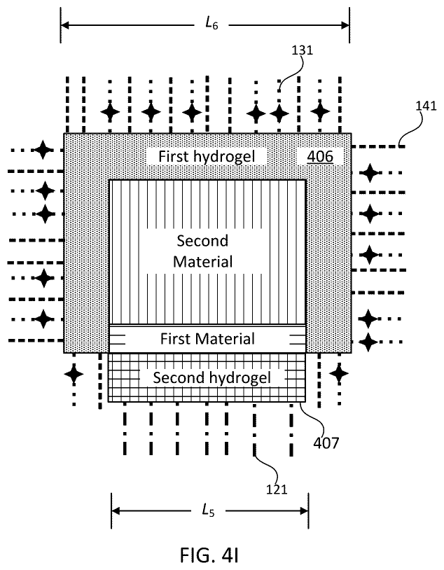

FIG. 3B illustrates a plan view of an alternative example particle 100 that is shaped as a polygon in the lateral dimension, e.g., approximately a square or rectangle. In this example, the first region 120 of the particle has a length L₃ in a first direction and a length La in a second direction, such that the surface area A₃ of the first region 120 in the lateral dimension may approximately be expressed as A₃=L₃L₄. Additionally, in this example, the second region 150 of the particle has a length L₁ in the first direction and a length L₂ in the second direction, such that the surface area A₁ of the second region 150 in the lateral dimension may approximately be expressed as A₁=L₁L₂. In other examples (not specifically illustrated), the first region 120 and the second region 150 may have any suitable respective shape(s). For example, the first region 120 may be substantially circular in the lateral dimension and the second region 150 may be substantially a polygon in the lateral dimension. Or, for example, the first region 120 may be substantially a polygon in the lateral dimension and the second region 150 may be substantially circular in the lateral dimension. In some examples, illustratively as described with reference to FIGS. 4A-4I, the first region 120 may have substantially the same lateral dimension(s) as the second region. That is, in certain examples of the configuration described with reference to FIG. 3B, L₁=L₃ and L₂=L₄; in these examples, D₁ may be larger than, L₁, L₂, L₃, and L₄. In certain examples of the configuration described with reference to FIG. 3A, D₂=D₃; in these examples, D₁ may be larger than both D₂ and D₃.

FIG. 3C illustrates a cross-section of the example particle 100 described with reference to FIG. 3A or with reference to FIG. 3B. In the example shown in FIG. 3C, the particle 100 may have a thickness T in a dimension orthogonal to the lateral dimension illustrated in FIG. 3A or 3B. In this example, the first region 120 of the particle 100 occupies only a portion of the thickness T, while the second region 150 of the particle is present through substantially the entire thickness T. In other examples described elsewhere herein, the second region 150 of the particle may occupy only a portion of the thickness T. In some examples described herein, particle 100 may be substantially spherical, such that thickness T substantially corresponds to the diameter of the particle. Although FIG. 3C illustrates the first region 120 as having diameter D₃ and the second region 150 as having diameter D₂, it will be appreciated that in examples in which one or both of these regions is a polygon the noted dimensions can correspond to lengths rather than to diameters of such region(s). The overall surface area of particle 100 in three dimensions, and the respective surface areas of first region 120 and second region 150 in three dimensions, can readily be determined.

As shown in FIGS. 3A-3C, polynucleotide 151, 151′ may be coupled to the first region 120 of particle 100 in a manner such as described with reference to FIG. 1B-1C or 2A-2B, illustratively through formation of duplex 161 illustrated in FIG. 1C or bonding pair such as described with reference to Table 1 or Table 2. Polynucleotide 151, 151′ may have a diameter of gyration 161 having dimension D₁ in the lateral dimension. Within this diameter of gyration 161, polynucleotide 151, 151′ may move (gyrate) in such a manner as to sufficiently occupy all of the space within that diameter that another polynucleotide may not be able to access the first region 120 of the particle and thus may not be captured. For example, as illustrated in FIG. 3C, captured polynucleotide 151, 151′ may sufficiently occupy the space surrounding first region 120 to inhibit adapters 121′ of any other polynucleotides (illustratively, polynucleotide 152, 152′ and polynucleotide 153, 153′) from being captured using the first region 120 of particle 100. As such, in some examples, first region 120 may be configured to have a dimension which is approximately equal to or smaller than a diameter of gyration of the polynucleotide.

While different polynucleotides may have different diameters of gyration than one another, particle 100, and the first region 120 thereof, may be configured and designed for use in capturing and amplifying polynucleotides that have a length that falls within a certain range of lengths (that is, that have a number of base pairs that falls within a certain range of number of base pairs). Illustratively, particle 100, and the first region 120 thereof, may be configured and sized for use in capturing and amplifying polynucleotides having a length of about 100 base pairs to about 1000 base pairs, e.g., a length of about 200 base pairs to about 600 base pairs. For example, particle 100 may configured and sized for use in a workflow that uses a library preparation method to fragment polynucleotides into lengths which fall within a defined range, and to couple adapters 154, 154′, 155, 155′ and 121′ to the resulting fragments. One skilled in the art would be able to determine the diameter of gyration of a given double-stranded polynucleotide using teachings otherwise known in the art, for example in Tree et al., “Is DNA a good model polymer?,” Macromolecules 46 (20): 8369-8382 (2013), the entire contents of which are incorporated by reference herein. Based on the teachings provided herein, one skilled in the art would be able to design and prepare a particle having a first region 120 with dimension(s) that are approximately equal to or smaller than the diameter of gyration of a polynucleotide with which the particle is to be used.

Although FIGS. 1A-1H illustrate an example in which a particle is used to capture a double-stranded polynucleotide, in other examples a particle may be used to capture a single-stranded polynucleotide.

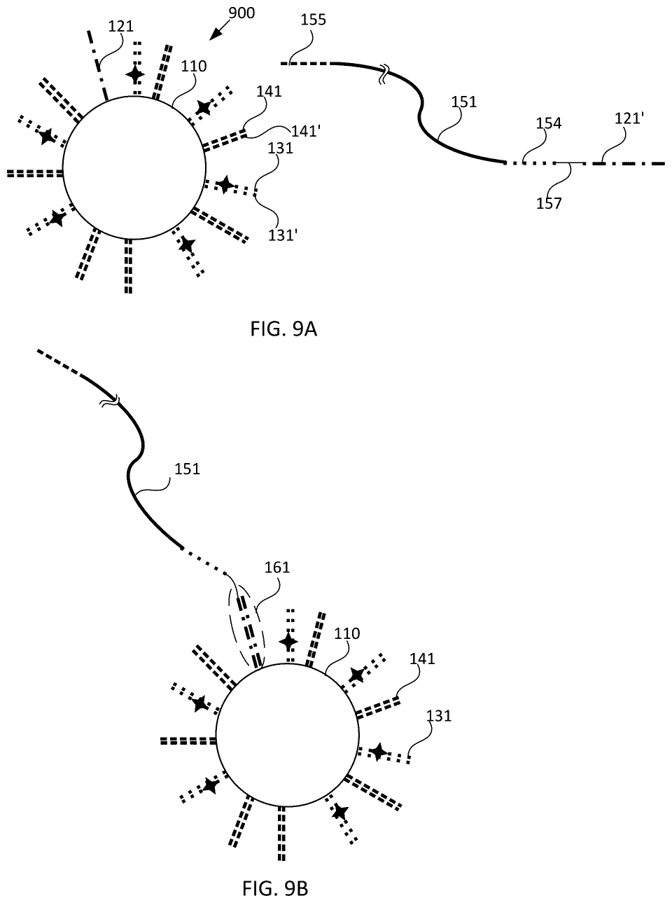

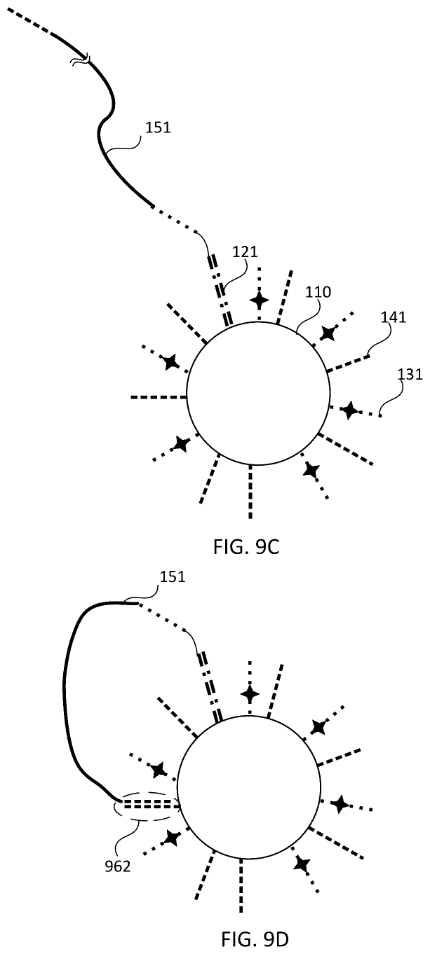

For example, FIGS. 9A-9D schematically illustrate additional example structures and operations for capturing and amplifying a polynucleotide using a particle. Referring now to FIG. 9A, particle 900 may be configured similarly as particle 100, e.g., may include core 100 to which may be coupled first and second amplification primers 131, 141, and seeding primer(s) 121 or a moiety 221 that may be used to react with a moiety which is coupled to the polynucleotide in a manner such as described with reference to FIGS. 2A-2B. The seeding primer(s) 121 or moiety 221 may be located in a first region 120 of particle 100, and the first and second amplification primers 131, 141 may be located in a second region of the particle in which a mixture of first and second amplification primers is located, e.g., in a manner such as described with reference to FIGS. 1A-1H. As illustrated in FIG. 9A, particle 900 also may include complementary oligonucleotides 131′ which are hybridized to respective first amplification primers 131, and complementary oligonucleotide 141′ which are hybridized to respective second amplification primers 141, when the polynucleotide is captured. The complementary oligonucleotides 131′, 141′ may be used to inhibit particle 900 from capturing any polynucleotides other than via seeding primer 121 (or moiety 221).

For example, referring now to FIG. 9B, particle 900 may be contacted, in the fluid, with single-stranded target polynucleotides, e.g., single-stranded polynucleotide fragments that are generated using commercially available fragmentation or tagmentation techniques, using DNA or RNA that it is desired to sequence. In the nonlimiting example illustrated in FIG. 9B, each of the polynucleotides 151 may include first and second adapters, e.g., first adapter 154 which is substantially complementary to amplification primer 131, e.g., is or includes cP5 (also referred to as P5′); and second adapter 155 which is substantially complementary to amplification primer 141, e.g., is or includes cP7 (also referred to as P7′). The single-stranded polynucleotides may include an additional single-stranded seeding adapter 121′ which may be configured similarly as described with reference to FIGS. 1A-1H, e.g., which is substantially complementary to seeding primer(s) 121, and which optionally is coupled to adapter 154 of polynucleotide 151 via linker 157. In some examples, optional seeding adapter 121 may include cPX (also referred to as PX′) or cPY (also referred to as PY′).

As shown in FIG. 9B, seeding adapter 121′ of single-stranded polynucleotide 151 may hybridize to seeding primer 121 to form duplex 161. Because adapters 154 and 155 are single-stranded, they would be able to hybridize to adapters 131 and 141 if oligonucleotides 131′, 141′ were not already hybridized to adapters 131 and 141, respectively. As such, oligonucleotides 131′ 141′ inhibit adapters 154, 155 of any other polynucleotides from being able to become hybridized to particle 900, and thus inhibit the particle from capturing any other polynucleotides. Accordingly, substantially all seeding of the polynucleotide may be expected to be via hybridization between seeding adapter 121′ and seeding primer 121 in the first region of the particle 100. After the initial hybridization described with reference to FIG. 9B, oligonucleotides 131′, 141′ then may be removed so that polynucleotide 151 may be amplified. More specifically, as illustrated in FIG. 9C, oligonucleotides 131′, 141′ respectively may be dehybridized from amplification primers 131, 141, e.g., by heating particle 900 to a temperature which is sufficiently high to dissociate oligonucleotide 131′ from primer 131 and to dissociate oligonucleotide 141′ from primer 141, while polynucleotide 151 remains coupled to the particle. For example duplex 161 may have a higher melting temperature than do the duplexes 131, 131′ and 141, 141′. Or, for example, polynucleotide 151 may be coupled to particle 900 via the reaction product between moieties in a manner such as described with reference to FIGS. 2A-2B.

After oligonucleotides 131′, 141′ are removed, polynucleotide 151 may be amplified using primers 131, 141. For example, as illustrated in FIG. 9D, polynucleotide 151 may bend such that adapter 155 hybridizes with one of primers 141 to form duplex 962 using bridge amplification or other suitable amplification process. An amplified cluster then may be formed using polynucleotide 151, for example by performing amplification operations any suitable number of times so as to substantially fill the second region of the particle with an at least functionally monoclonal cluster, and in some examples a substantially monoclonal cluster, e.g., with amplicons of target polynucleotide 151. For example, amplicons within the second region of the particle may include at least about 60% amplicons of one selected target polynucleotide, or at least about 70% amplicons of one selected target polynucleotide, or at least about 80% amplicons of one selected target polynucleotide, or at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide. The resulting particle may be used in a manner such as described with reference to FIGS. 1F-1H.

From the foregoing disclosure, it will be appreciated that primers and adapters having any suitable sequences may be used. In one nonlimiting example, amplification primers 131 are P5 amplification primers, and amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc. Seeding primers 121 may have any suitable sequence which is orthogonal to the sequences of amplification primers 131, 141. In some examples, seeding primers 121 may be PX primers having the sequence AGGAGGAGGAGGAGGAGGAGGAGG (SEQ ID NO: 5). In other examples, seeding primers 121 may be PY primers having the sequence 5′-GAA GAA GAA GAA GAA GAA GAA GAA GAA GAA-3′ (SEQ ID NO: 6). In examples in which seeding primers 121 are PX primers, seeding adapters 121′ may be cPX (also referred to as PX′) adapters having the sequence CCTCCTCCTCCTCCTCCTCCTCCT (SEQ ID NO: 7). In examples in which seeding primers 121 are PY primers, seeding adapters 121′ may be cPY (also referred to as PY′) adapters having the sequence 5′-TTC TTC TTC TTC TTC TTC TTC TTC TTC TTC-3′ (SEQ ID NO: 8).

It will be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may further facilitate the amplification of a single target polynucleotide per particle and the production of a substantially monoclonal population of amplicons on a particle. For example, the rate of amplification of the first captured target polynucleotide within the second region of the present particles may be more rapid relative to much slower rates of transport and capture of target polynucleotides at the first region of the present particles. As such, the first target polynucleotide captured in the first region may be amplified rapidly and fill the entire second region, which may, in combination with other operations described herein, further inhibit the amplification of additional target polynucleotide(s) in the second region. Alternatively, even if a second target polynucleotide is captured at the particle after the first polynucleotide, the relatively rapid amplification of the first polynucleotide may fill enough of the second region to result in a signal that is sufficiently strong to perform sequencing by synthesis (e.g., the second region may be at least functionally monoclonal). The use of exclusion amplification may also result in super-Poisson distributions of monoclonal second regions; that is, the fraction of second regions in a collection of particles that are functionally monoclonal may exceed the fraction predicted by the Poisson distribution. Increasing super-Poisson distributions of useful clusters is useful because more functionally monoclonal particles may result in higher quality signal, and thus improved SBS.

Another method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification (ExAmp) works by using recombinase to facilitate the invasion of primers (e.g., primers attached to a substrate region) into double-stranded DNA (e.g., a target polynucleotide) when the recombinase mediates a sequence match. The present particles and methods may be adapted for use with recombinase to facilitate the invasion of the present amplification primers and orthogonal amplification primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For still further examples of amplification methods that are compatible with the present particles, see International Patent Application No. PCT/US2022/053005 to Ma et al., filed Dec. 15, 2022 and entitled “Hybrid Clustering,” and U.S. patent application Ser. No. 18/193,480 to Ma et al., filed Mar. 30, 2023 and entitled “Paired-End Resynthesis Using Blocked P5 Primers,” the entire contents of each of which are incorporated by reference herein.

### Methods of Making Particles for Use in Capturing and Amplifying Polynucleotides

It will be appreciated that a wide variety of particle geometries and configurations are compatible with operations such as described with reference to FIGS. 1A-1H, 2A-2B, 3A-3C, and 9A-9D. For example, FIGS. 4A-4I, 5A-5K, 6A-6M, 7A-7G, 8A-8C, 10A-10J, and 11A-11E schematically illustrate additional example particles for capturing and amplifying polynucleotides in a manner such as described with reference to FIGS. 1A-1H, 2A-2B, 3A-3C, and 9A-9D, and example flows of operations for preparing such particles.

In some examples, the capture primers (e.g., 131, 141, and/or 121) and/or moieties 221 optionally may be coupled to respective hydrogel(s) that are disposed on the particle. The hydrogel(s) themselves may be covalently coupled to the particle, or alternatively may be non-covalently coupled to the particle. Some examples herein relate to particles having different configurations of hydrogels. Such particles optionally may be used in operations such as described elsewhere herein, but it should be appreciated that such particles may be used in other types of operations besides those expressly disclosed herein.

FIGS. 4A-4I schematically illustrate an example flow of operations for preparing a particle that may be used to capture and amplify a polynucleotide. As illustrated in FIG. 4A, structure 400 may include substrate 401, lift-off layer 402, first material 403 a portion of which may eventually form a portion of one of the present particles, second material 404 a portion of which may eventually form a portion of that particle, and mask layer 405. Lift-off layer 402 may be disposed on substrate 401, first material 403 may be disposed on lift-off layer 402, second material 404 may be disposed on first material 403, and mask layer 405 may be disposed on second material 404. Non-limiting examples of materials suitable for use in substrate 401 include glass or other material which is substantially insoluble in organic solvents and resistant to dry etch. Non-limiting examples of materials suitable for use in lift-off layer 402 include photoresist and poly(methyl methacrylate) (PMMA). Non-limiting examples of materials suitable for use in first material 403 include chromium (Cr), aluminum (A₁), and copper (Cu). Non-limiting examples of materials suitable for use in second material 404 include a resin, tantalum oxide (TaOₓ), and silicon dioxide (SiO₂). A nonlimiting example of a resin suitable for use in second material 404 is a nano-imprint lithography (NIL) resin, such as a polymethylglutaride-based resin commercially available from Kayaku Advanced Materials, Inc. Non-limiting examples of materials suitable for use in mask layer 405 include chromium, aluminum, and copper.

As illustrated in FIG. 4B, mask layer 405 may be patterned, e.g., photolithographically using a photoresist (e.g., AZ series positive resist from Merck) or using NIL (e.g., using mr-NIL series from Kayaku Advanced Materials, Inc. In some examples, the lateral dimensions L₅ of patterned mask layer 405 illustrated in FIG. 4B may correspond to the lateral dimensions of both the first region 120 and the second region 150 such as described with reference to FIGS. 3A-3C. For example, L₅ illustrated in FIG. 4B may approximately correspond to L₁ and L₃ or to L₂ and La in configurations such as described with reference to FIG. 3B, or L₅ may approximately correspond to D₂ and D₃ in configurations such as described with reference to FIG. 3A. In these and other examples, the patterned mask layer may be used to mask respective portions of the first material and second material during an etch operation that removes unmasked portions of the first material and second material in a manner such as illustrated in FIG. 4C. In some examples, the etch operation may include one or more dry etch and/or wet etch operations. A wet etch operation for first material 403 may include, for example, use of a suitable Cr, Al, or Cu etchant appropriate to the composition of first material 403; a dry etch operation for first material 403 may include, for example, Cl₂ plasma. A dry etch operation for second material 404 may include, for examples in which the second material is a NIL resin, use of a CF₄ plasma or a mixture of 90% CF₄ and 10% O₂ plasma; or for examples in which the second material is TaOₓ or SiO₂, use of CHF₃ or a CHF₃+Ar plasma. The patterned mask layer 405 then optionally may be removed. Dry etch operations for layer 405 may include, for example, use of a chlorine-based plasma (e.g., BCl₃ and/or Cl₂). Alternatively, layer 405 may be wet etched, for example with potassium hydroxide (KOH), tetramethylammonium hydroxide (TMAH), nitric acid, acetic acid, or phosphoric acid. When removed as a sacrificial layer, when layer 405 includes aluminum it may be removed in acidic or basic conditions; when layer 405 includes copper it may be removed using an iodine and iodide solution or FeCl₃; or when layer 405 includes gold it may be removed using an iodine and iodide solution.

As illustrated in FIG. 4D, functional groups 412 may be coupled to the second material 404, for example via silane-containing bridges. The process of forming these may be referred to as “silanization.” Optionally, functional groups 412 also may be coupled to the first material, lift-off layer, and/or substrate. As illustrated in FIG. 4E, hydrogel 406 optionally may be disposed over the first and second materials 403, 404 that are patterned using operations such as described with reference to FIGS. 4A-4C. In some examples, the hydrogel may include a polyacrylamide, such as PAZAM or SFA. The hydrogel includes one or more functional groups which may be of the same type as one another, or may be of different types than one another. The functional group(s) of the hydrogel may react with functional groups 412 so as to couple to the hydrogel to the second material 404. For example, functional group 412 may include a norbornene, a cyclooctyne, or a bicyclononyne, and a functional group of hydrogel 406 may include an azide. For nonlimiting examples of silanizing, pairs of functional groups which may be used to couple a silane to a hydrogel, and for nonlimiting examples of hydrogels including functional groups, see U.S. Pat. No. 10,975,210 to Berti et al., the entire contents of which are incorporated by reference herein.

In a manner such as illustrated in FIG. 4F, while the hydrogel 406 is disposed over at least the patterned second material and optionally over the patterned first material, the portion of the assembly including the hydrogel and the patterned first and second materials may be removed, to form a particle 400′ that may be further processed to form particle 100 in a manner such as will be described below. Such a process may be referred to as “lift-off” because the particle 400′ may be removed substantially intact from substrate 401. Such lift-off may be performed using a suitable solvent or combination of solvents that dissolve lift-layer 402 or otherwise detach particle 400′ from substrate 401, such as a mixture of dimethyl sulfoxide (DMSO) and water (e.g., 90% DMSO in water); DMSO with sonication; acetone; propylene glycol methyl ether acetate (PGMEA); or an N-methyl-2-pyrrolidone (NMP) based stripper. As illustrated in FIG. 4G, the newly exposed surface of the first material then may be silanized in a manner such as described with reference to FIG. 4D (e.g., using the same type of functional group or a different type of functional group as added in FIG. 4D). As illustrated in FIG. 4H, a second hydrogel 407 may be coupled to the silanized first material. The second hydrogel 407 may be of the same type as first hydrogel 406, or may be of a different type than the first hydrogel. For example, the second hydrogel 407 may include a different type of functional group than the first hydrogel, so that different primers may be attached to the second hydrogel than to the first hydrogel. Any suitable operations may be used to inhibit the second hydrogel 407 from becoming disposed over first hydrogel 406. For example, deposition of second hydrogel 407 may be performed under high ionic strength (e.g., in the presence of 10x PBS, NaCl, KCl, or the like), such that the second hydrogel 407 substantially does not deposit on or adhere to the first hydrogel 406. As such, the bare portion of first material illustrated in FIG. 4G selectively receives the second hydrogel 407.

Seeding primers 121 or moieties 221 may be coupled to second hydrogel 407 at any suitable time. For example, as illustrated in FIG. 4I, primers 121 (or moieties 221, not specifically illustrated) may be coupled directly or indirectly to second hydrogel 407 via reaction between an appropriate functional group within hydrogel 407 and an appropriate functional group coupled to the respective primer 121 or moiety 221. Additionally, amplification primers 131, 141 may be coupled to first hydrogel 406 at any suitable time. For example, primers 131, 141 may be coupled to functional groups within hydrogel 406 in a manner similar to that described for primers 121 or moieties 221. The resulting particle 40 illustrated in FIG. 4I corresponds to particle 100 described with reference to FIGS. 1A-1H. In this example, because hydrogel 406 forms part of the lateral dimension of the second region of the particle, the second region may have a lateral dimension L₆ illustrated in FIG. 4I and either corresponding to D₂ in FIG. 3A, with D₃ of the first material being smaller than D₂ (L₆); or corresponding to L₁ or L₂ in FIG. 3B, with L₃ and/or La being smaller than L₁ or L₂ (L₆). L₅ illustrated in FIG. 4I may approximately correspond to L₃ and/or L₄ in configurations such as described with reference to FIG. 3B, or L₅ may approximately correspond to D₃ in configurations such as described with reference to FIG. 3A. Particles 40 may be used as particle 100 in a manner such as described with reference to FIG. 1A-1H, 2A-2B, 3A-3C, or 9A-9D to capture and amplify a polynucleotide.

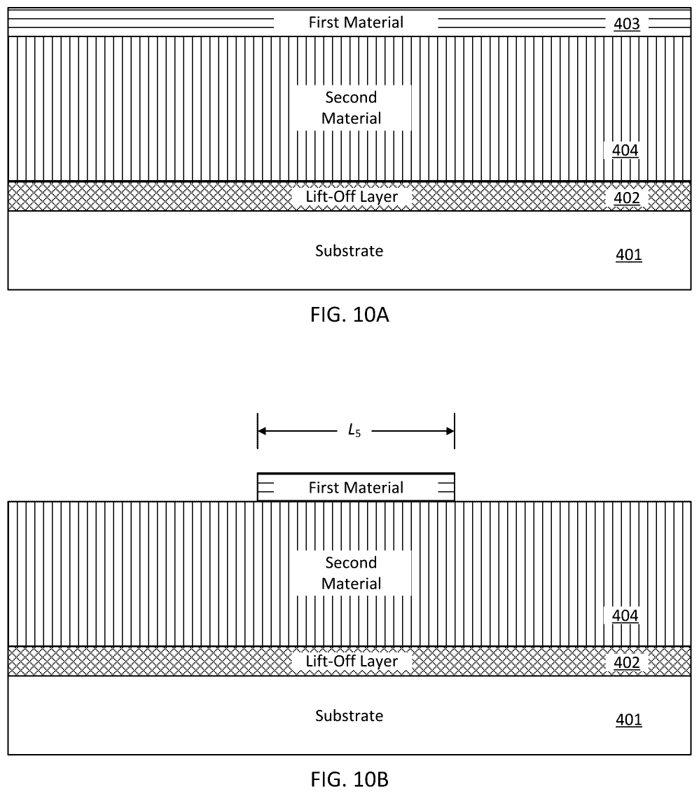

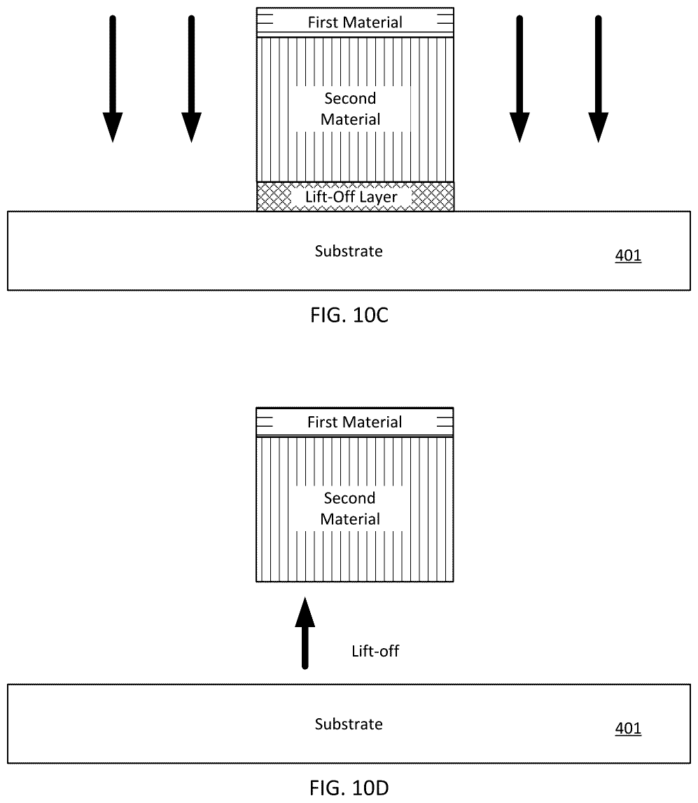

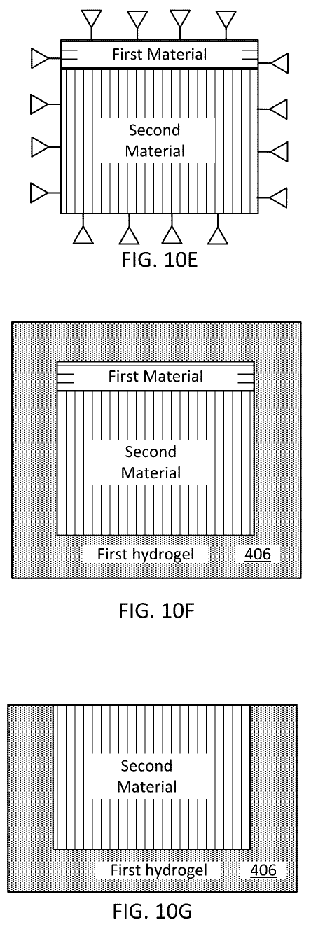

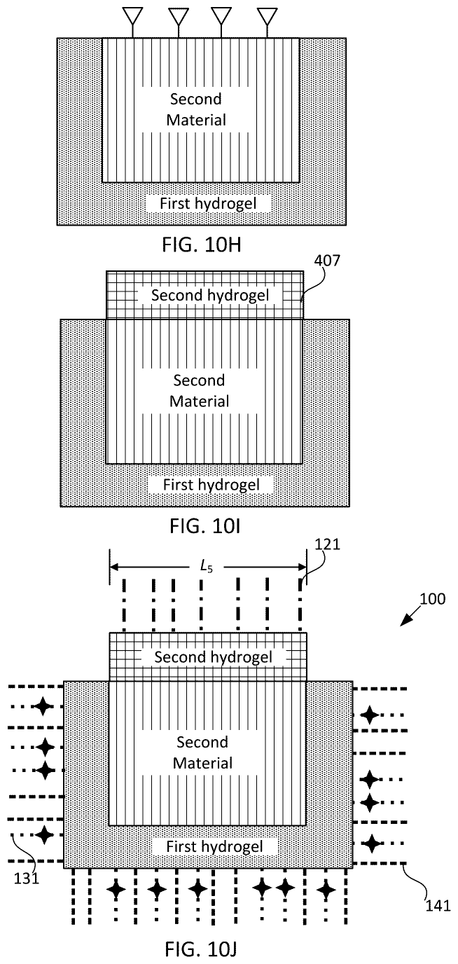

Note that the relative positions of the first material and second material optionally may be reversed relative to that described with reference to FIGS. 4A-4I. Additionally, the particle optionally may be detached from the substrate before disposing one or both of the hydrogels thereon. For example, FIGS. 10A-10J schematically illustrate another example flow of operations for preparing a particle that may be used to capture and amplify a polynucleotide. The flow of operations may begin with the structure illustrated in FIG. 10A, which is similar to that described with reference to FIG. 4A except that the second material 404 is disposed on the lift-off layer 402, the first material 403 is disposed on the second material, and the mask layer 405 is omitted (looked at another way, first material 403 is used as a mask layer). Here, the options for the second material 404, lift-off layer 402, and substrate 401 may be the same as described with reference to FIG. 4A. The first material 403 may include aluminum, in some examples.

As illustrated in FIG. 10B, the first material may be patterned similarly as the mask layer described with reference to FIG. 4B. As illustrated in FIG. 10C, the second material, and lift-off layer may be patterned using the first material and any suitable combination of wet etch and/or dry etch steps using reactants which are suitable to the materials forming such layers. As illustrated in FIG. 10D, the patterned first material and second material then may be detached from the substrate using lift-off similarly as described with reference to FIG. 4F, except that such lift-off is performed before depositing the first hydrogel. As illustrated in FIG. 10E, the second material (and optionally also the first material) may be silanized in a manner such as described with reference to FIG. 4D. As illustrated in FIG. 10F, first hydrogel 406 may be coupled to the silanized portions of the particle (e.g., at least the second material) in a similar manner as described with reference to FIG. 4E. As illustrated in FIG. 10G, the first material may be removed (e.g., dissolved), for example using a suitable weak base solution (such as diluted KOH and tetramethyl ammonium hydroxide (TMAH) and/or AZ400K. As illustrated in FIG. 10H, the resulting exposed region of the second material may be silanized in a manner such as described with reference to FIG. 4G. As illustrated in FIG. 10I, second hydrogel 407 then may be coupled to the silanized second material in a manner such as described with reference to FIG. 4H. Any suitable operations may be used to inhibit the second hydrogel 407 from becoming disposed over first hydrogel 406, e.g., such as described with reference to FIG. 4G. As illustrated in FIG. 10J, primers 121 (or moieties 221) may be coupled to the second hydrogel 407 at any suitable time, and primers 131, 141 may be coupled to the first hydrogel 406 at any suitable time, in a manner such as described with reference to FIG. 4I.

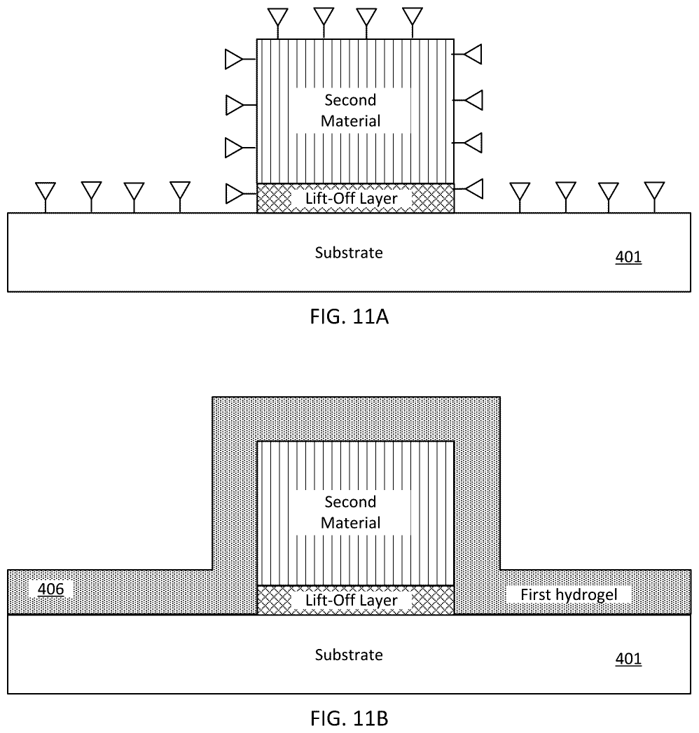

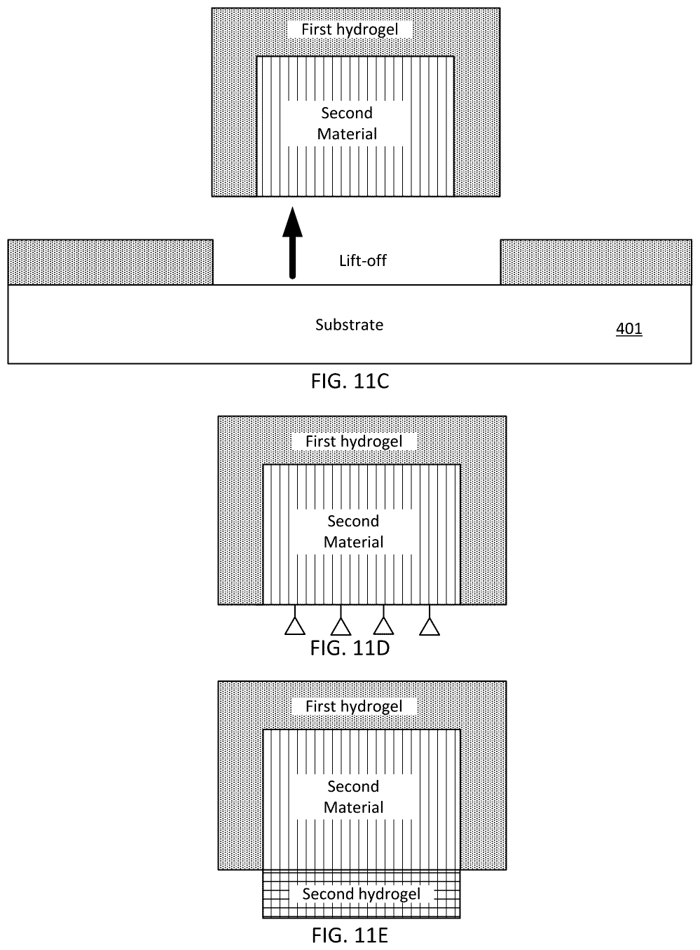

As another example, FIGS. 11A-11E schematically illustrate another example flow of operations for preparing a particle that may be used to capture and amplify a polynucleotide. The flow of operations may begin with the structure illustrated in FIG. 11A, which is similar to that described with reference to FIG. 10C except that the first material (or etch mask) is removed after patterning second material and lift-off layer. Here, the options for the second material 404, lift-off layer 402, and substrate 401 may be the same as described with reference to FIG. 4A. The first material 403 may include Cr, A₁, or Cu, in some examples. As illustrated in FIG. 11A, the second material may be silanized in a manner such as described with reference to FIG. 4D. As illustrated in FIG. 11B, first hydrogel 406 may be coupled to the silanized portions of the particle (e.g., at least the second material) in a similar manner as described with reference to FIG. 4E. As illustrated in FIG. 11C, the second material with first hydrogel disposed thereon may be removed from the substrate using lift-off similarly as described with reference to FIG. 4F. As illustrated in FIG. 11D, the resulting exposed region of the second material may be silanized in a manner such as described with reference to FIG. 4G. As illustrated in FIG. 11E, second hydrogel 407 then may be coupled to the silanized second material in a manner such as described with reference to FIG. 4H. Any suitable operations may be used to inhibit the second hydrogel 407 from becoming disposed over first hydrogel 406, e.g., such as described with reference to FIG. 4G. Primers 121 (or moieties 221) may be coupled to the second hydrogel 407 at any suitable time, and primers 131, 141 may be coupled to the first hydrogel 406 at any suitable time, in a manner such as described with reference to FIG. 4I.

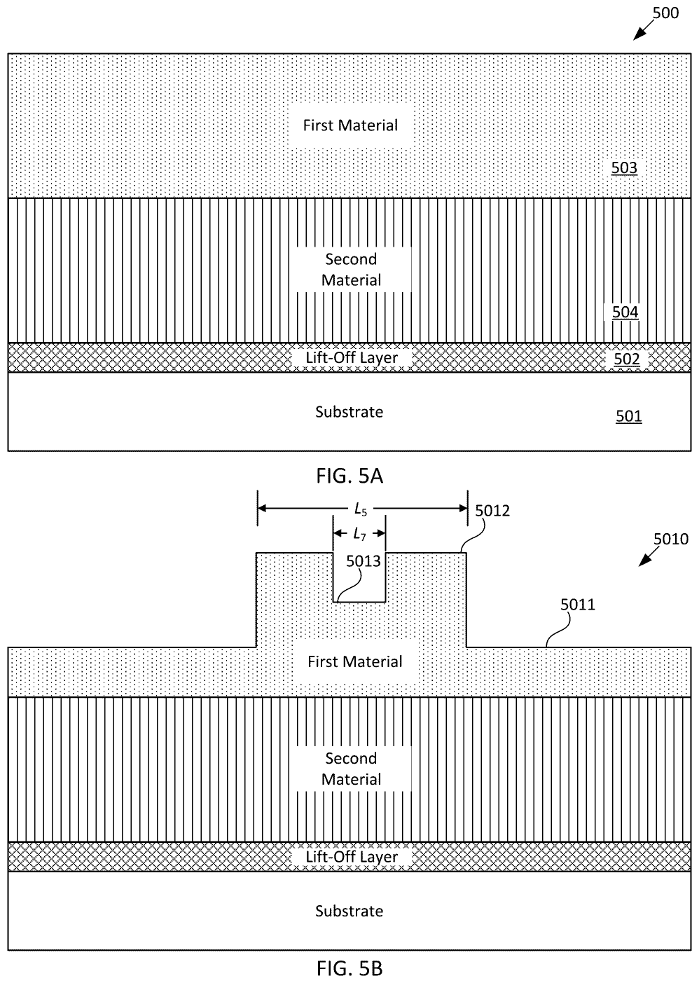

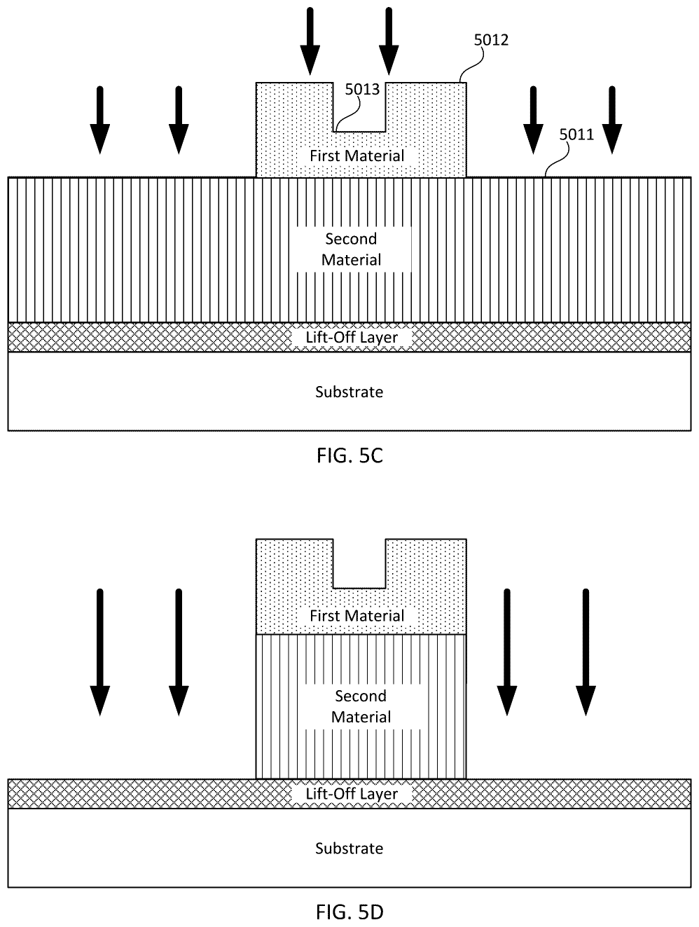

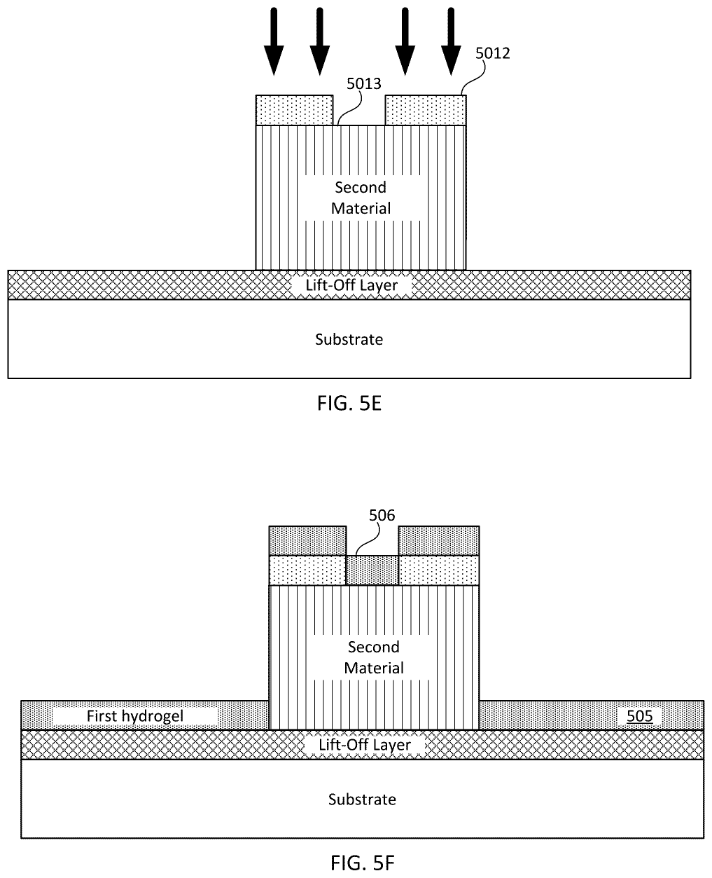

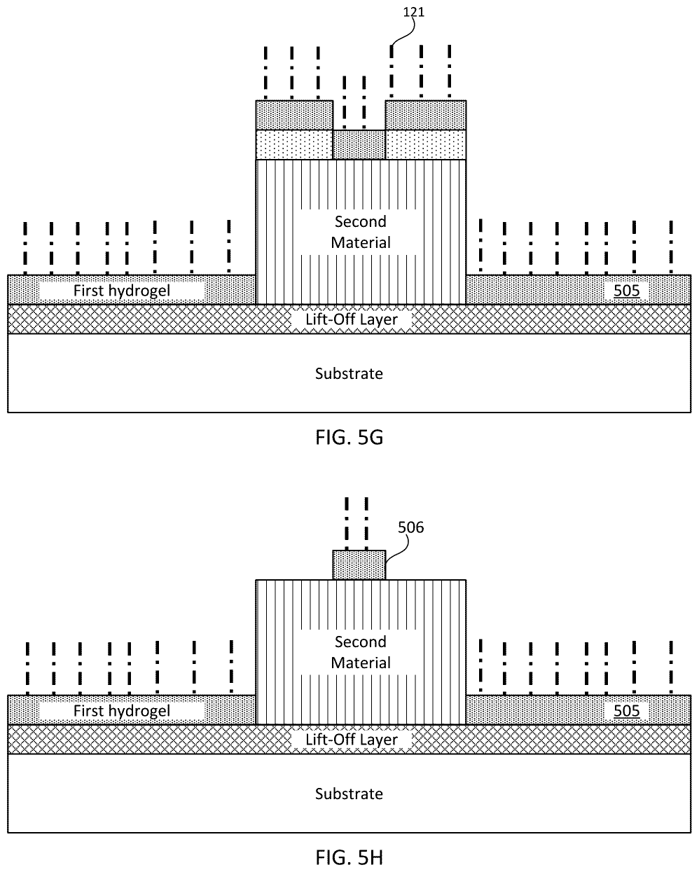

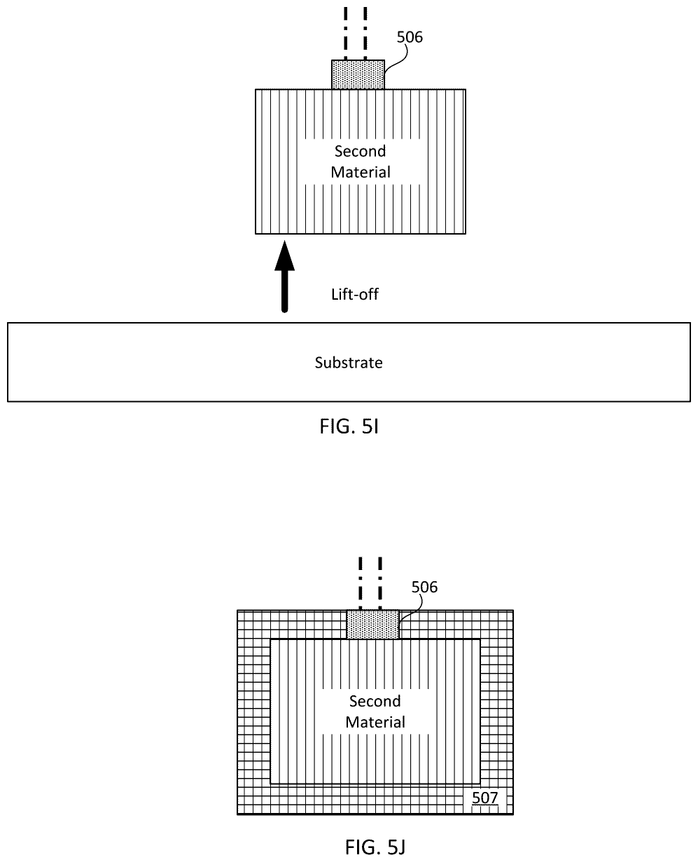

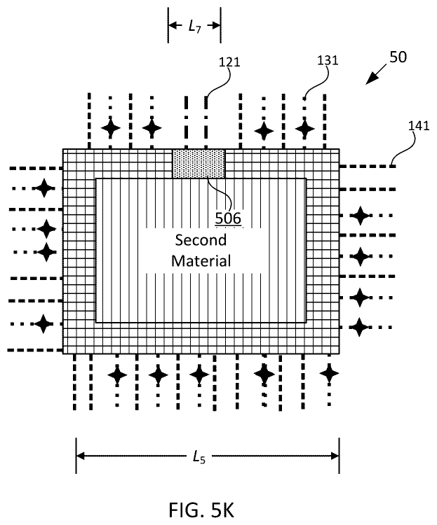

FIGS. 5A-5K schematically illustrate another example flow of operations for preparing a particle that may be used to capture and amplify a polynucleotide. As illustrated in FIG. 5A, structure 500 may include substrate 501, lift-off layer 502, first material 503 a portion of which may be used to pattern a hydrogel (not illustrated in FIG. 5A, but described below) to form the first region 120 of a particle, and second material 504 a portion of which may be used to define the second region 150 of a particle. Lift-off layer 502 may be disposed on substrate 501, second material 504 may be disposed on lift-off layer 502, and first material 503 may be disposed on second material 504. Non-limiting examples of materials suitable for use in substrate 501, lift-off layer 502, and second material 504 are described with reference to FIGS. 4A-4I. Non-limiting examples of materials suitable for use in first material 503 include a NIL resist (such as mr-NIL series from Kayaku).

As illustrated in FIG. 5B, a stepped pattern 5010 may be impressed into first material 503, for example using NIL. More specifically, stepped pattern 5010 may include a first region 5011, a second region 5012, and a third region 5013. As illustrated in FIG. 5B, the second region 5012 and the third region 5013 each may be raised relative to the first region 5011. The third region 5013 may be lower than, located within, and substantially surrounded by, the second region 5012. The second region 5012 may be located within, and substantially surrounded by, the first region 5011. In some examples, the lateral dimensions L₅ of second region 5012 illustrated in FIG. 5B may correspond to the lateral dimensions of second region 150 such as described with reference to FIGS. 3A-3C. For example, L₅ illustrated in FIG. 5B may approximately correspond to L₁ or to L₂ in configurations such as described with reference to FIG. 3B, or L₅ may approximately correspond to D₂ in configurations such as described with reference to FIG. 3A. In some examples, the lateral dimensions L₇ of third region 5013 illustrated in FIG. 5B may correspond to the lateral dimensions of first region 120 such as described with reference to FIGS. 3A-3C. For example, L₇ illustrated in FIG. 5B may approximately correspond to L₃ or to La in configurations such as described with reference to FIG. 3B, or L₅ may approximately correspond to D₃ in configurations such as described with reference to FIG. 3A.

As illustrated in FIG. 5C, a first etching operation may be used to remove a portion of the first material such that the lower surface of the first region 5011 is substantially located at the upper surface of the second material, while the second and third regions 5012 and 5013 remain above the upper surface of the second material. As illustrated in FIG. 5D, the remaining portion of the first material then is used as an etch mask to protect the underlying portion of the second material during a second etching operation. As illustrated in FIG. 5E, the first material then is further etched in a third etching process may be used to remove another portion of the first material such that the lower surface of the third region 5013 is substantially located at the upper surface of the second material, while the second region remains above the upper surface of the second material. In some examples, the first, second, and third etching processes may include respective dry etch operations. Dry etch operations for materials 503 and 504 are described elsewhere herein.

A hydrogel 505 then may be formed within the third region 5013. For example, in a manner such as illustrated in FIG. 5F, the hydrogel may be disposed over the structure resulting from the operations described with reference to FIGS. 5A-5E. Patterned region 506 of the hydrogel 505 may be disposed on the patterned second material 504, and may be coupled thereto using silanization such as described elsewhere herein. In a manner such as illustrated in FIG. 5G, primers 121 (or moieties 221) may be grafted to first hydrogel 505 in a manner such as described elsewhere herein. In a manner such as illustrated in FIG. 5H, the patterned first material may be removed, leaving region 506 with primers 121 (or moieties 221) coupled thereto coupled to second material 506. As illustrated in FIG. 5I, the second material may be detached from the substrate using lift-off, using a suitable solvent or combination of solvents that dissolve lift-layer 502, some examples of which are provided with reference to FIGS. 4A-4I. Exposed portions of second material then may be silanized in a manner such as described elsewhere herein, and coupled to a second hydrogel 507 in a manner such as illustrated in FIG. 5J. Any suitable operations may be used to inhibit the second hydrogel 507 from becoming disposed over first hydrogel 506, e.g., such as described with reference to FIG. 4G.

Amplification primers 131, 141 may be coupled to second hydrogel 507 at any suitable time, for example, as illustrated in FIG. 5K. In this example, L₅ illustrated in FIG. 5K may approximately correspond to L₁ or to L₂ in configurations such as described with reference to FIG. 3B, or L₅ may approximately correspond to D₂ in configurations such as described with reference to FIG. 3A. L₇ illustrated in FIG. 5K may approximately correspond to L₃ or to L₄ in configurations such as described with reference to FIG. 3B, or L₅ may approximately correspond to D₃ in configurations such as described with reference to FIG. 3A. Particles 50 illustrated in FIG. 5K may be used as particle 100 in a manner such as described with reference to FIGS. 1A-1H, 2A-2B, 3A-3C, and 9A-9D to capture and amplify a polynucleotide.

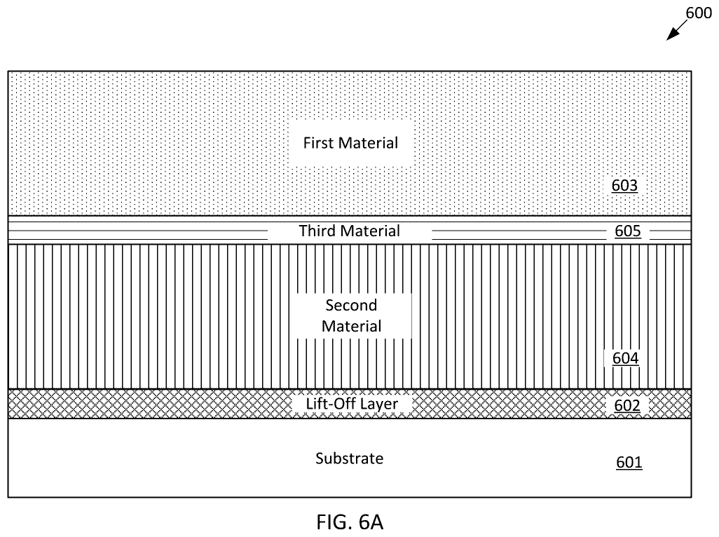

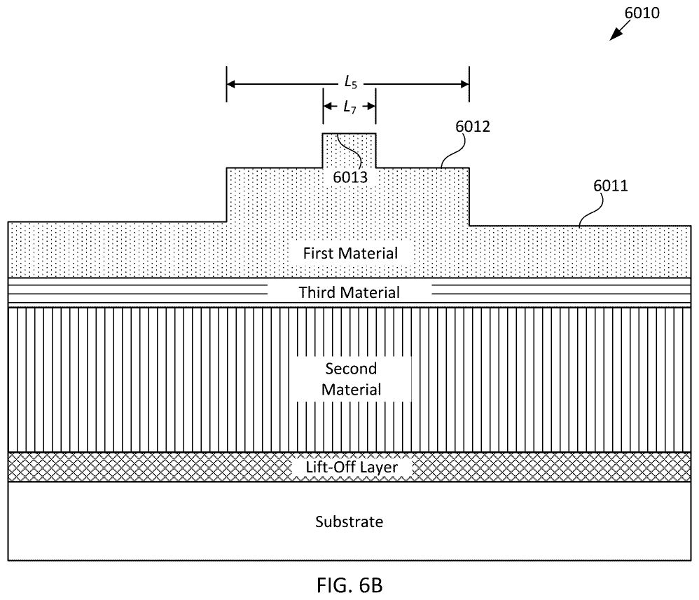

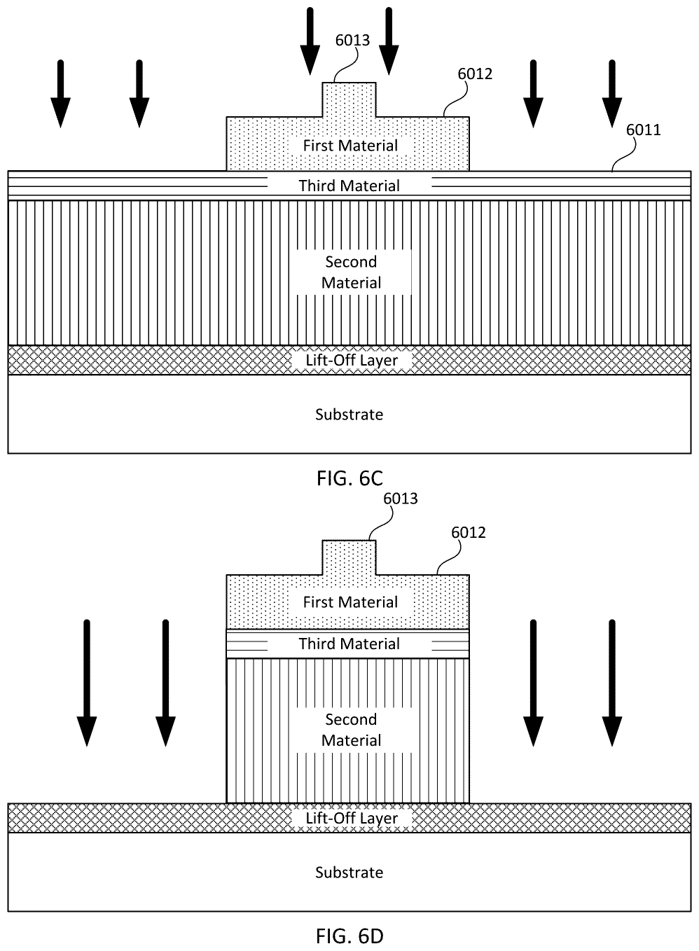

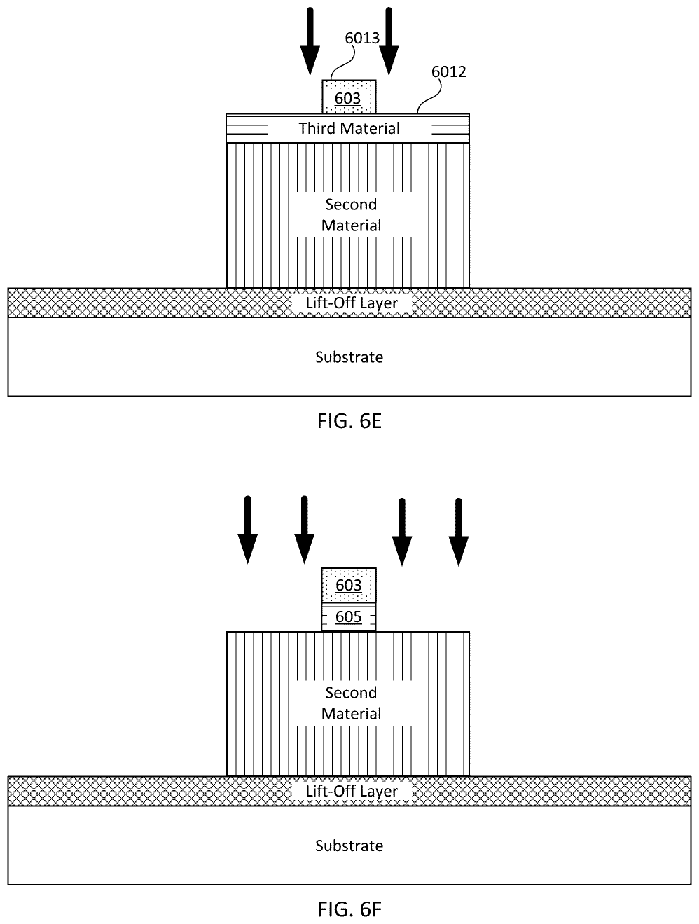

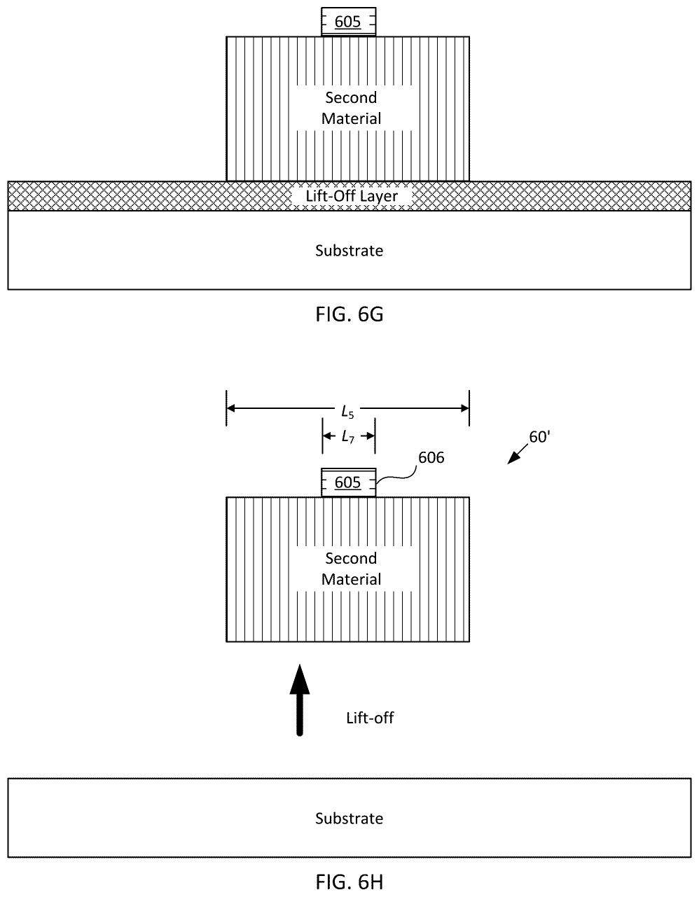

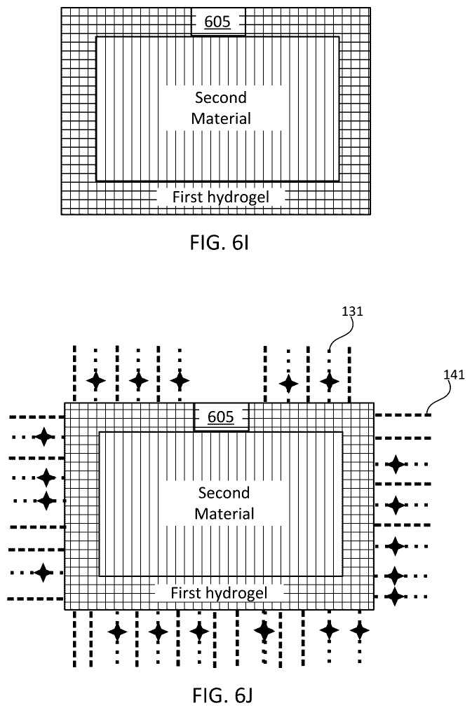

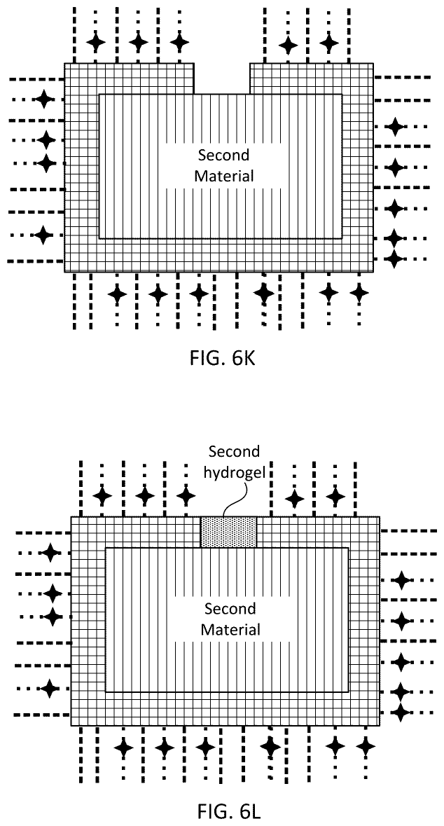

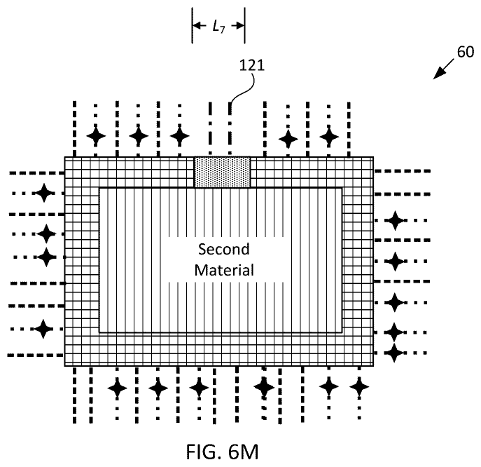

FIGS. 6A-6M schematically illustrate another example flow of operations for preparing a particle that may be used to capture and amplify a polynucleotide. As illustrated in FIG. 6A, structure 600 may include substrate 601, lift-off layer 602, first material 603 a portion of which may be used to pattern third material 605 to form the first region 120 of a particle, and second material 604 a portion of which may eventually form the second region 150 of a particle. Lift-off layer 602 may be disposed on substrate 601, second material 604 may be disposed on lift-off layer 602, third material 605 may be disposed on second material 604, and first material 603 may be disposed on third material 605. Non-limiting examples of materials suitable for use in substrate 601, lift-off layer 602, and second material 604 are described with reference to FIGS. 4A-4I. Non-limiting examples of materials suitable for use in first material 603 are described with reference to first material 503 of FIGS. 5A-5K. Non-limiting examples of third material 605 are described with reference to first material 403 of FIGS. 10A-10J.

As illustrated in FIG. 6B, a stepped pattern 6010 may be impressed into first material 603, for example using NIL. More specifically, stepped pattern 6010 may include a first region 6011, a second region 6012, and a third region 6013. As illustrated in FIG. 6B, the second region 6012 and the third region 6013 each may be raised relative to the first region 6011. The third region 6013 may be higher than, located within, and substantially surrounded by, the second region 6012. The second region 6012 may be located within, and substantially surrounded by, the first region 6011. In some examples, the lateral dimensions L₅ of second region 6012 illustrated in FIG. 6B may correspond to the lateral dimensions of second region 150 such as described with reference to FIGS. 3A-3C. For example, L₅ illustrated in FIG. 6B may approximately correspond to L₁ or to L₂ in configurations such as described with reference to FIG. 3B, or L₅ may approximately correspond to D₂ in configurations such as described with reference to FIG. 3A. In some examples, the lateral dimensions L₇ of third region 6013 illustrated in FIG. 6B may correspond to the lateral dimensions of first region 120 such as described with reference to FIGS. 3A-3C. For example, L₇ illustrated in FIG. 6B may approximately correspond to L₃ or to La in configurations such as described with reference to FIG. 3B, or L₅ may approximately correspond to D₃ in configurations such as described with reference to FIG. 3A.

As illustrated in FIG. 6C, a first etching operation may be used to remove a portion of the first material such that the lower surface of the first region 6011 is substantially located at the upper surface of the third material, while the second and third regions 6012 and 6013 remain above the upper surface of the third material. For example, an O₂ plasma may be used to dry etch the NIL resist of first material 603. As illustrated in FIG. 6D, the remaining portion of the first material then is used as an etch mask to protect the underlying portion of the third material and the second material during a second etching operation. For example, a Cl₂ plasma may be used to dry etch the third material 6011. In examples in which the second material is a resin, a CF₄ or CF₄+O₂ plasma may be used to etch it; in examples in which the second material is TaOₓ or SiO₂, a CHF₃ or CHF₃+O₂ plasma. As illustrated in FIG. 6E, the first material 603 then is further etched in a third etching process (e.g., another O₂ plasma dry etch) which may be used to remove another portion of the first material such that the lower surface of the second region 6012 is substantially located at the upper surface of the third material, while the third region 6013 remains above the upper surface of the third material. As illustrated in FIG. 6F, the third material then is further etched in a fourth etching process (e.g., using Cl₂ plasma) that uses the remaining portion of the first material 603 as an etch mask to protect the underlying portion of the third material 605. As illustrated in FIG. 6G, the remaining portion of the first material 603 then is removed (e.g., using O₂ plasma). As illustrated in FIG. 6H, a structure including the patterned second material and patterned third material is removed from the substrate to form particle 60′. The particle then may be silanized and a first hydrogel disposed on the particle, as illustrated in FIG. 6I. The first hydrogel may be disposed on the second material, and optionally also may be disposed on the third material (not illustrated). As illustrated in FIG. 6J, amplification primers 131 and 141 then may be coupled to the first hydrogel in a manner such as described elsewhere herein. As illustrated in FIG. 6K, the third material then may be removed, e.g., using lift-off with a weak base solution. As illustrated in FIG. 6L, within the gap left by the removal of the third material, the second material may be silanized and a second hydrogel may be disposed thereon, e.g., in a manner similar to that described with reference to FIG. 4H. In a manner such as illustrated in FIG. 6M, seeding primers 121 or moieties 221 may be coupled to the second hydrogel. L₇ illustrated in FIG. 6M may approximately correspond to L₃ or to La in configurations such as described with reference to FIG. 3B, or L₇ may approximately correspond to D₃ in configurations such as described with reference to FIG. 3A. Particles 60 illustrated in FIG. 6M may be used as particle 100 in a manner such as described with reference to FIG. 1A-1H, 2A-2B, 3A-3C, or 9A-9D to capture and amplify a polynucleotide.

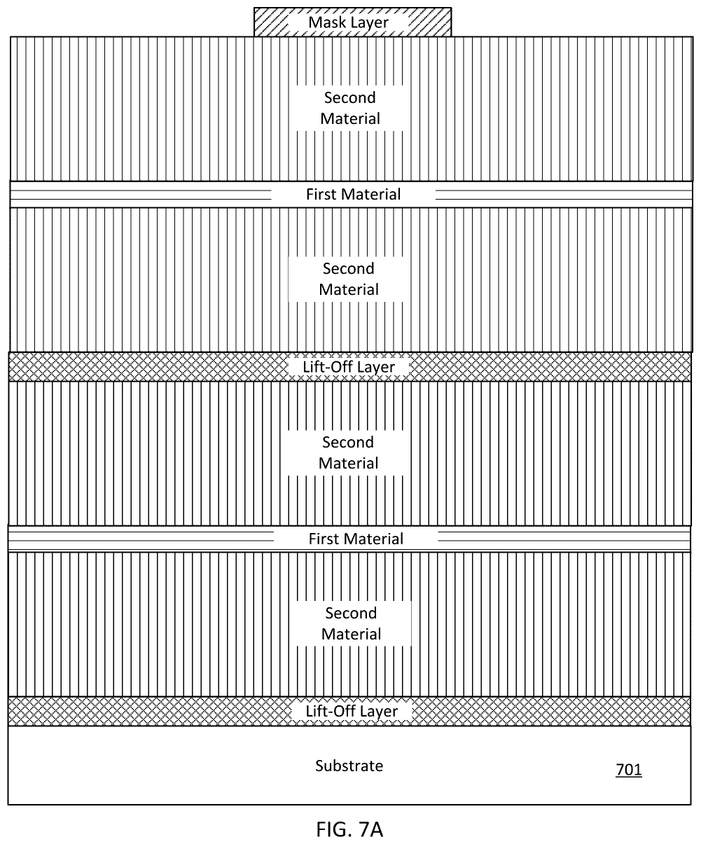

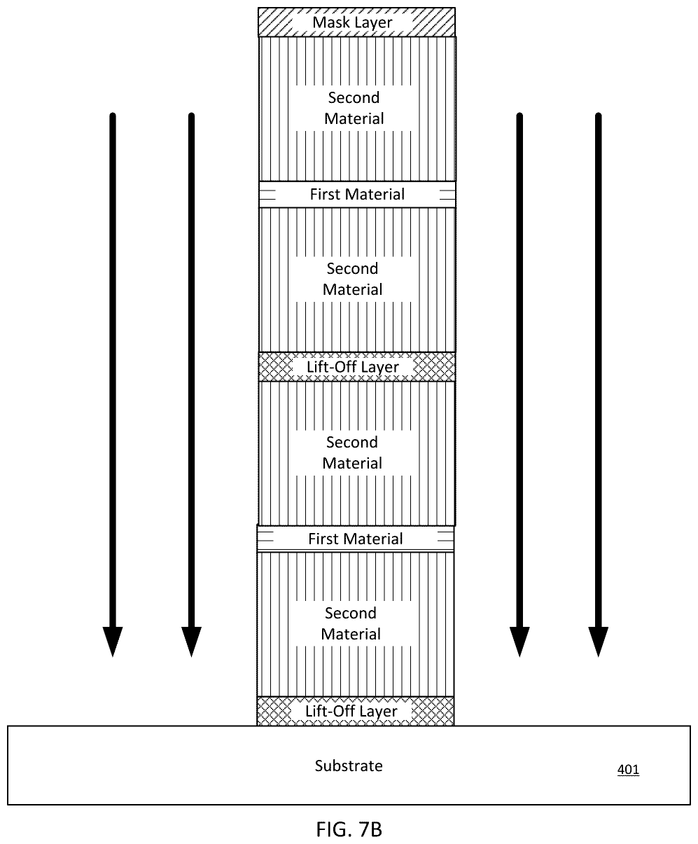

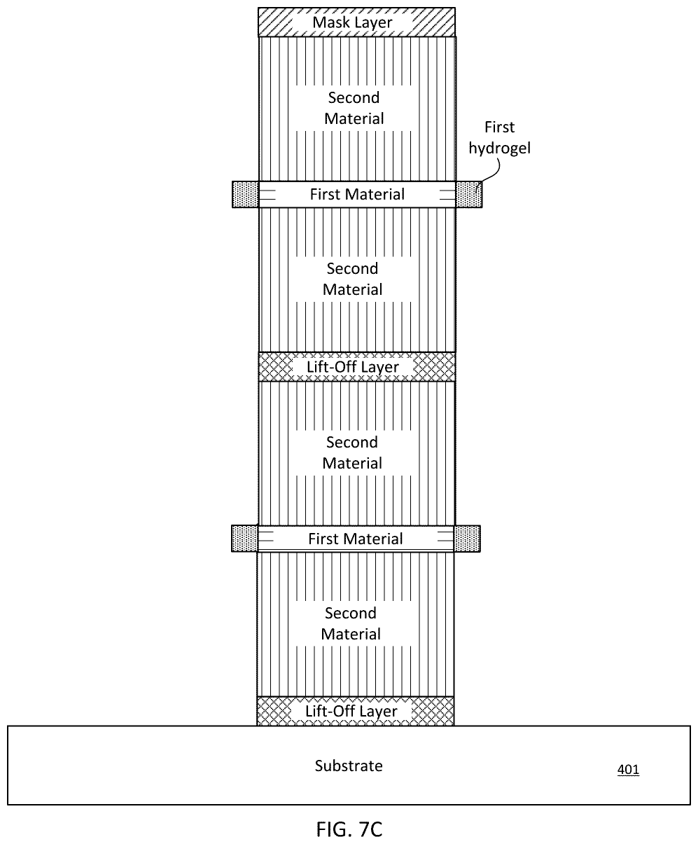

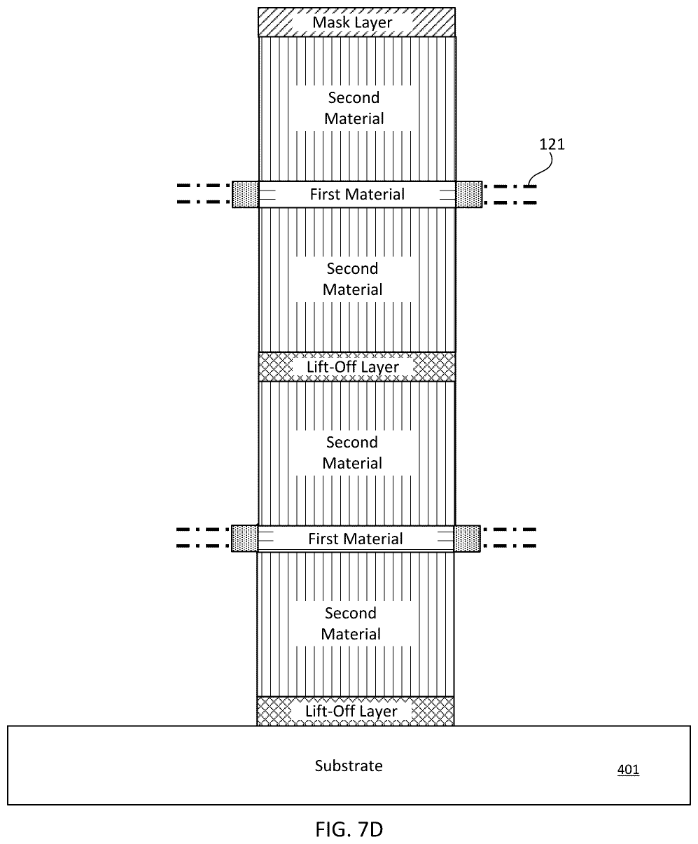

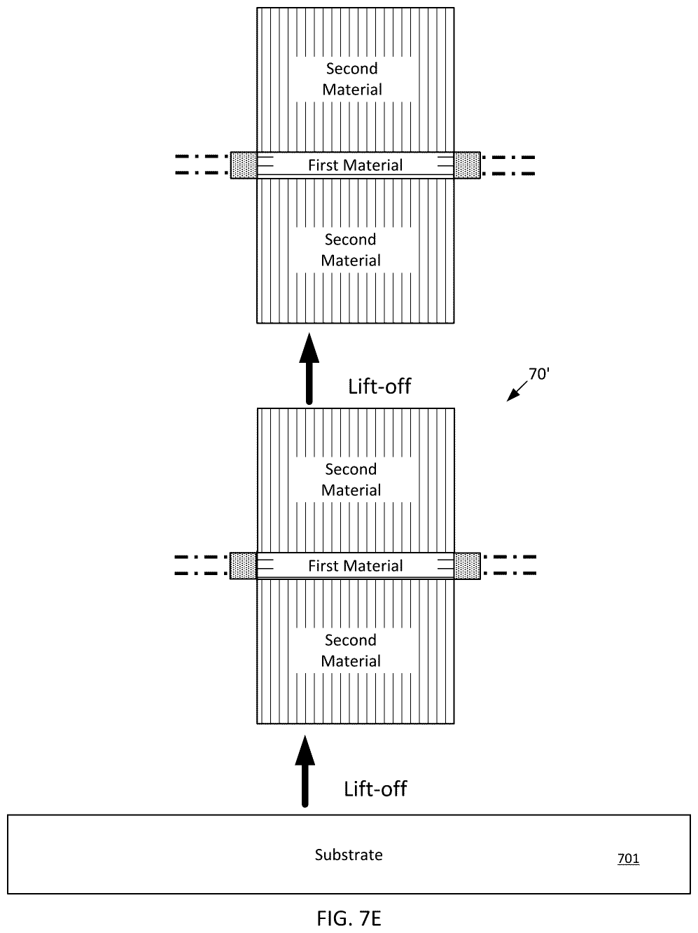

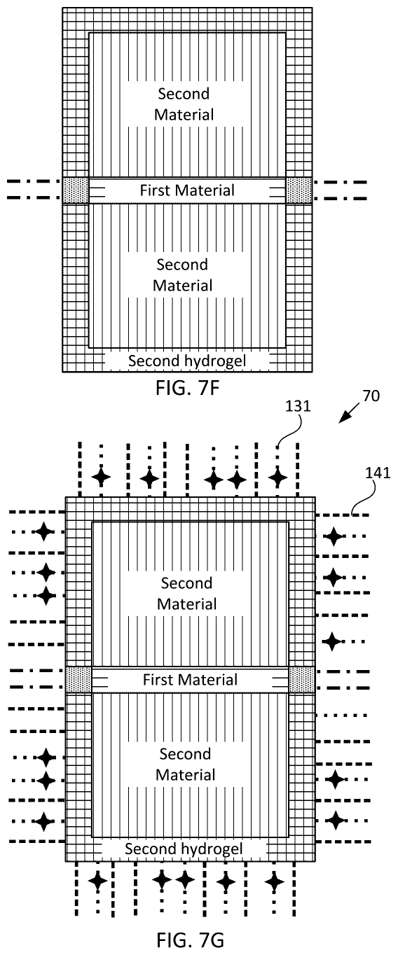

FIGS. 7A-7G schematically illustrate another example flow of operations for preparing a particle that may be used to capture and amplify a polynucleotide. The structure illustrated in FIG. 7A may include multiple “sandwich” structures each including a first material which is disposed between first and second layers of a second material. A first one of the sandwich structures may be disposed on a lift-off layer which is disposed on the substrate, and additional ones of the sandwich structures may be disposed thereon and separated from one another by additional lift-off layers. Although only two such sandwich structures are illustrated in FIG. 7A, it will be appreciated that any suitable number of sandwich structures (e.g., one, two, three, four, five, or more than five) structures may be included. A patterned mask layer may be disposed over the stack of sandwich structures. Example materials and methods for patterning masks are provided elsewhere herein. As illustrated in FIG. 7B, the stack of sandwich structures may be patterned using etching, in which the mask layer acts as an etch stop. Illustratively, the second material may include TaOₓ, which in some examples may be etched using CHF₃ or CHF₃+Ar plasma; the first material may include NIL resin, which in some examples may be etched using CF₄ or CF₄+O₂ plasma; and the lift-off layer may include photoresist or PMMA, which in some examples may be etched using O₂ plasma. As illustrated in FIG. 7C, a first hydrogel then may be disposed on the first material, e.g., following an ashing process which promotes adhesion of the first hydrogel to the first material (e.g., NIL resin). As illustrated in FIG. 7D, seeding primers 121 (or moieties 221, not illustrate) may be coupled to the first hydrogel in a manner such as described elsewhere herein. As illustrated in FIG. 7E, a lift-off process then may be used to detach particles 70′ from the substrate and from one another. As illustrated in FIG. 7F, the second material may be silanized and a second hydrogel disposed thereon. As illustrated in FIG. 7G, amplification primers 131, 141 may be coupled to the second hydrogel in a manner such as described elsewhere herein. Particles 70 illustrated in FIG. 7G may be used as particle 100 in a manner such as described with reference to FIG. 1A-1H, 2A-2B, 3A-3C, or 9A-9D to capture and amplify a polynucleotide. The “capture” material of particle 70 occupies about 10% or less of the surface area of the particle, thus inhibiting capture of more than one polynucleotide using the particle.

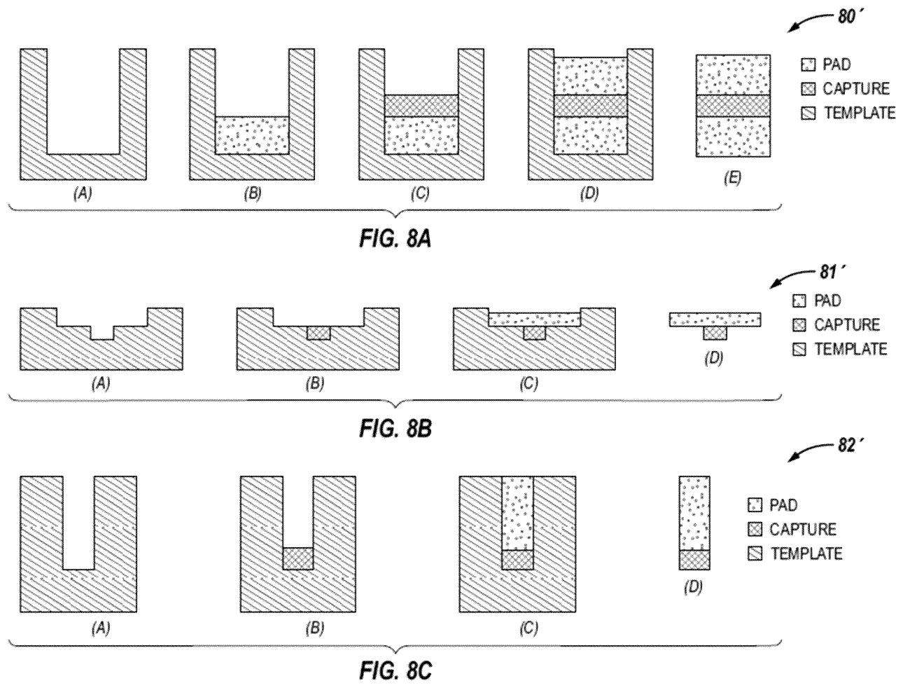

FIGS. 8A-8C schematically illustrate additional respective example flows of operations for preparing a particle that may be used to capture and amplify a polynucleotide. Turning first to FIG. 8A, operation (A) includes preparing a template within which a particle may be prepared. For example, the template may include an unreactive material, such as perfluoropolyether elastomer, that is patterned using any suitable operations. In this example, the template includes a recess of substantially uniform depth. Operation (B) of FIG. 8A includes disposing a first material (e.g., a material corresponding to second material 404 described with reference to FIGS. 4A-4I, illustratively a first hydrogel or other polymer) within the template. Operation (C) of FIG. 8A includes disposing a second material (e.g., a material corresponding to first material 403 described with reference to FIGS. 4A-4I, illustratively a second hydrogel or other polymer) within the template, on top of the first material. Operation (D) of FIG. 8A includes disposing additional first material (e.g., additional material corresponding to second material 404 described with reference to FIGS. 4A-4I, illustratively the first hydrogel or other polymer) within the template, on top of the second material. Operation (E) of FIG. 8A includes releasing particle 80′ from the template, for example using an etch step. Primers 121 or moieties 221 may be coupled to the second material (denoted “capture” in FIG. 8A), and primers 131, 141 may be coupled to the first material (denoted “pad” in FIG. 8A) to form particles which may be used in a manner such as described with reference to FIG. 1A-1H, 2A-2B, 3A-3C, or 9A-9D to capture and amplify a polynucleotide. The “capture” material of particle 80′ occupies about 10% or less of the surface area of the particle, thus inhibiting capture of more than one polynucleotide using the particle.

Turning now to FIG. 8B, operation (A) includes preparing a template within which a particle may be prepared, in a similar manner as described with reference to FIG. 8A. In this example, the template includes a stepped recess including a deeper portion surrounded by a shallower portion. Operation (B) of FIG. 8B includes disposing a first material (e.g., a material corresponding to second material 404 described with reference to FIGS. 4A-4I, illustratively a first hydrogel or other polymer) within the deeper portion of the template. Operation (C) of FIG. 8B includes disposing a second material (e.g., a material corresponding to first material 403 described with reference to FIGS. 4A-4I, illustratively a second hydrogel or other polymer) within the template, on top of the first material, in the shallower portion of the template. Operation (D) of FIG. 8B includes releasing particle 81′ from the template, for example using an etch step. Primers 121 or moieties 221 may be coupled to the second material, and primers 131, 141 may be coupled to the first material to form particles which may be used in a manner such as described with reference to FIG. 1A-1H, 2A-2B, 3A-3C, or 9A-9D to capture and amplify a polynucleotide.

Turning now to FIG. 8C, operation (A) includes preparing a template within which a particle may be prepared, in a similar manner as described with reference to FIG. 8A. In this example, the template includes a recess of substantially uniform depth. Operation (B) of FIG. 8C includes disposing a first material (e.g., a material corresponding to second material 404 described with reference to FIGS. 4A-4I, illustratively a first hydrogel or other polymer) within the deeper portion of the template. Operation (C) of FIG. 8C includes disposing a second material (e.g., a material corresponding to first material 403 described with reference to FIGS. 4A-4I, illustratively a second hydrogel or other polymer) within the template, on top of the first material, in the shallower portion of the template. Operation (D) of FIG. 8C includes releasing particle 82′ from the template, for example using an etch step. Primers 121 or moieties 221 may be coupled to the second material, and primers 131, 141 may be coupled to the first material to form particles which may be used in a manner such as described with reference to FIG. 1A-1H, 2A-2B, 3A-3C, or 9A-9D to capture and amplify a polynucleotide.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method for capturing and amplifying a polynucleotide, the method comprising:
capturing the polynucleotide at a particle comprising a first region and a second region,
the first region comprising a first moiety that captures the polynucleotide,
the second region comprising a plurality of amplification primers and having a surface area which is substantially larger than the surface area of the first portion; and
using the plurality of amplification primers to amplify the captured polynucleotide.

2. The method of claim 1, wherein the captured polynucleotide inhibits capture of any other polynucleotide at the first region of the particle.

3. The method of claim 1, wherein the surface area of the first portion inhibits capture of more than one polynucleotide.

4. The method of claim 1, wherein the first region has a dimension which is approximately equal to or smaller than a diameter of gyration of the polynucleotide.

5. The method of claim 1, wherein the surface area of the first region is approximately 20 nm² or less.

6. The method of claim 1, wherein the first region forms less than about 10% of an overall surface area of the particle.

7. The method of claim 1, wherein the first region is formed using a first material, and the second region is formed using a second material that is different than the first material.

8. The method of claim 7, wherein at least one of the first and second regions comprises a hydrogel.

9. The method of claim 1, wherein the first moiety comprises a capture primer having a sequence that is orthogonal to sequences of the amplification primers.

10. The method of claim 9, wherein the polynucleotide comprises a single-stranded primer that is substantially complementary to, and hybridizes to, the capture primer.

11. The method of claim 1, wherein the polynucleotide comprises at least one adapter which is complementary to an amplification primer of the plurality of amplification primers.

12. The method of claim 1, wherein the first moiety covalently or non-covalently bonds to a second moiety coupled to the polynucleotide.

13. The method of claim 1, wherein the plurality of amplification primers comprises a first type of amplification primers and a second type of amplification primers having a sequence that is orthogonal to a sequence of the first type of amplification primers.

14. The method of claim 13, wherein the first type of amplification primers comprises an excision moiety.

15. The method of claim 1, wherein the captured polynucleotide is double stranded.

16. The method of claim 15, wherein the captured polynucleotide is amplified using strand invasion.

17. The method of claim 1, wherein the captured polynucleotide is single stranded.

18. The method of claim 17, wherein the captured polynucleotide is amplified using bridge amplification.

19. The method of claim 17, wherein the particle further comprises oligonucleotides which are hybridized to respective amplification primers when the polynucleotide is captured.

20. The method of claim 1, further comprising contacting the particle with a substrate.

21. The method of claim 20, further comprising electrostatically attracting the particle to the substrate.

22. The method of claim 20, wherein the particle is contacted with the substrate after capturing the polynucleotide with the particle.

23. The method of claim 20, wherein the particle is contacted with the substrate after amplifying the captured polynucleotide.

24. A method of sequencing a polynucleotide, the method comprising:
capturing and amplifying the polynucleotide using the method of claim 1 to generate amplicons of the polynucleotide coupled to the particle;
capturing the particle in a region of a flowcell; and
sequencing the amplicons in the region of the flowcell.

25. A particle for seeding and amplifying a polynucleotide, the particle comprising:
a first region comprising a first moiety to capture the polynucleotide; and
a second region comprising a second material having a surface area which is substantially larger than the surface area of the first portion, and comprising a plurality of amplification primers.

26-37. (canceled)


SEQUENCE LISTING (13 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg

SEQ ID NO: 5 - DNA, 24 bp, synthetic construct
  source 1..24: mol_type = other DNA; note = PX primer
aggaggaggaggaggaggaggagg

SEQ ID NO: 6 - DNA, 30 bp, synthetic construct
  source 1..30: mol_type = other DNA; note = PY primer
gaagaagaagaagaagaagaagaagaagaa

SEQ ID NO: 7 - DNA, 24 bp, synthetic construct
  source 1..24: mol_type = other DNA; note = cPX adapter
cctcctcctcctcctcctcctcct

SEQ ID NO: 8 - DNA, 30 bp, synthetic construct
  source 1..30: mol_type = other DNA; note = cPY adapter
ttcttcttcttcttcttcttcttcttcttc

SEQ ID NO: 9 - protein, 13 aa, synthetic construct
  source 1..13: note = SpyTag amino acid sequence
AHIVMVDAYKPTK

SEQ ID NO: 10 - protein, 134 aa, synthetic construct
  source 1..134: note = SpyCatcher amino acid sequence
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 11 - protein, 5 aa, synthetic construct
  source 1..5: note = Sortase-coupling example moiety 221 or 221 prime
LPXTG

SEQ ID NO: 12 - protein, 4 aa, synthetic construct
  source 1..4: note = Sortase-coupling example moiety 221 prime or 221
GGGG

SEQ ID NO: 13 - protein, 5 aa, synthetic construct
  source 1..5: note = Sortase-coupling example moiety 221 prime or 221
GGGGG